(19) United States
(12) Patent Application Publication
(10) Pub. No.: US 2022/0195415 A1
(43) Pub. Date: Jun. 23, 2022

(54) Nucleic Acid Constructs and Methods for Their Manufacture

(71) Applicant: LIGHTBIO LIMITED, Hampton (GB)

(72) Inventors: Thomas Antony James ADIE, Hampton (GB); Paul James ROTHWELL, Hampton (GB); Michal LEGIEWICZ, Hampton (GB)

(21) Appl. No.: 17/605,474

(22) PCT Filed: Apr. 23, 2020
(86) PCT No.: PCT/GB2020/051003

§ 371 Date: Oct. 21, 2021

(30) Foreign Application Priority Data
Apr. 23, 2019 (GB) 1905651.4

(51) Int. Cl. C12N 15/10; C12Q 1/6806

(52) U.S. Cl. CPC C12N 15/10; C12Q 2531/125; C12Q 2525/301; C12Q 1/6806

(57) ABSTRACT

The present invention concerns new artificially synthesized single stranded nucleic acid molecules which may be used in many applications, and templates and methods for making the same. There are a multitude of uses for single stranded nucleic acid molecules, including but not limited to vectors for the delivery of sequences (for example a gene sequence, or a template for gene editing, gene knock-in or knock-down) or in bioengineering, for example as for constructing highly ordered materials from nanoparticle building blocks.

### FIELD OF THE INVENTION

The present invention concerns new artificially synthesized single stranded nucleic acid molecules which may be used in many applications, and templates and methods for making the same. There are a multitude of uses for single stranded nucleic acid molecules, including but not limited to vectors for the delivery of sequences (for example a gene sequence, or a template for gene editing, gene knock-in or knock-down) or in bioengineering, for example as for constructing highly ordered materials from nanoparticle building blocks. Single stranded nucleic acids can take various geometries, and can provide a function, for example aptamers and nucleic acid enzymes. If the single stranded nucleic acid is used as a vector these may be used to transfer nucleic acid sequences/fragments to a target cell, either directly or encapsulated by further components.

### BACKGROUND TO THE INVENTION

There is an increasing appreciation for the functions that nucleic acids assume within a cell, above and beyond coding for the production of proteins. Double stranded structures by their very nature have been studied extensively, but it should be appreciated that these can form rigid assemblies in the cell due to the base-pairing between complementary nucleotides. The most flexible regions of nucleic acids are often non-base paired and include single stranded deoxyribose nucleic acid (ssDNA) and ribonucleic acid (ssRNA) regions that are involved in vital processes within the cell. For instance, double stranded DNA (dsDNA) is unwound by enzymes such as DNA polymerase, exposing ssDNA sections. These sections are then available for transcription into ssRNA, such as messenger RNA (mRNA), or for interaction with other proteins that recognise the ssDNA.

Single stranded nucleic acid molecules are of interest to those skilled in the art of delivering nucleic acid to cells in particular, since the nucleic acid is immediately available within the transfected cell, and does not require “unwinding” by an appropriate enzyme to expose the relevant genetic information (for example for transcription and translation or insertion into the genome). They are considered to be an optimal delivery vector for several applications, not least gene transfer, gene editing and biosensing. Another potential application is the provision of DNA vaccines. Alternatively, the single stranded DNA may have a function related to its conformation, i.e. as an aptamer. However, longer single stranded nucleic acids, for example in the range of thousands of nucleotides in length, are currently inefficiently or inaccurately produced, limiting their utility, as discussed further below. The most common method of producing long oligonucleotides is cloning of the sequence into plasmid for cultivation in bacteria, followed by restriction digestion and purification of dsDNA sequence which is then strand-stripped to produce ssDNA. Notwithstanding the issues with bacterial propagation, there are many inefficiencies and purification issues. The entire plasmid backbone sequence is amplified and must then be separated and discarded, along with the bacterial genome. The stripping of the secondary strand to reveal the single stranded nucleic acid molecule yet further decreases the efficiency by another fifty percent.

In order to take advantage of the therapeutic potential of single stranded nucleic acid, there is required a method of manufacture which is efficient and scalable, making quantities of material that are on a commercial scale. Current techniques are limited in their ability to scale-up to produce materials in a cost-effective, accurate, quick and safe manner. It is also desirable to accurately produce single stranded molecules that are 200 nucleotides in length or more.

Both ssDNA and dsDNA donor sequences can act as efficient gene-editing templates, but the choice of donor construct is often dictated by the length of the sequence to be introduced. ssDNA donors have been mostly used for applications requiring small edits, mostly because generating longer ssDNA has been found to be problematic as discussed above. ssDNA templates have been found to have a unique advantage in terms of repair specificity when used in gene editing (Design and specificity of long ssDNA donors for CRISPR-based knock-in Han Li, Kyle A. Beckman, Veronica Pessino, Bo Huang, Jonathan S. Weissman, Manuel D. Leonetti bioRxiv 178905), and therefore their use is desirable.

By its very nature, linear single stranded nucleic acid is quickly degraded within cells, since free 3′ and 5′ ends are available for enzyme such as single strand nucleases, which “chew back” the ends and destroy the nucleic acid. Therefore, there is a need to provide a stabilised single stranded nucleic acid construct for such purposes, wherein the free 3′ and 5′ ends are protected from immediate degradation.

Many viral vectors used to deliver genetic material to cells have a single stranded genome, either as RNA or DNA, and therefore there is a precedent for the use of single stranded nucleic acid in gene delivery.

For example, adeno-associated virus (AAV) is an interesting gene therapy vehicle, and belongs to the parvovirus family and in nature is dependent on co-infection with other viruses (such as adenovirus), in order to replicate. AAV is essentially a proteinaceous shell surrounding a single-stranded DNA genome of about 4.7 kilobases (kb). There are hundreds of unique AAV strains. Its single-stranded genome contains, inter alia, Rep (Replication) and Cap (Capsid) genes. These coding sequences are flanked at both termini by inverted terminal repeats (ITRs) which are usually 145 nucleotides long.

Recombinant AAV (rAAV), which lacks viral DNA, is essentially ITR-flanked transgenes protected in a protein-based nanoparticle engineered for DNA cargo delivery into the nucleus of a cell. The main consideration in the design of such a rAAV vector is the packaging size of the transgene and associated sequences between the two ITRs. 5 kb (including the viral ITRs) appears to be the current limit in order to ensure that the ITR-flanked transgene is packaged. Alternatively, the ITR flanked transgene (or other sequence of interest) could be introduced directly into a cell without packaging, meaning that the “artificial genome” could indeed be longer.

Typically used nucleic acid molecules in the art, such as gene delivery vectors derived from viral genomes may be problematic as they can induce an immune response in the recipient of the gene delivery vector, since the immune system can recognise the circulating “foreign” DNA. If DNA is produced in bacterial cells, it will have prokaryotic patterns of DNA methylation which may be identified as foreign within eukaryotic organisms, and similarly rejected. For example, plasmids (pDNA) are circular dsDNA molecules which are naturally occurring, extra chromosomal DNA fragments stably inherited from one generation to the next. Plasmids and derivatives thereof have been used as gene delivery vectors with varied amounts of success.

The method of producing the nucleic acid vectors may also be problematic. Manufacturing nucleic acid structure within bacterial cells risks the contamination of the final product with lipopolysaccharides (LPS), endotoxins and other prokaryotic-specific molecules. These have the capability to raise an immune response in eukaryotic organisms, since they are effectively an indicator of a microbial pathogen. Indeed, manufacturing nucleic acid vectors within any cell-based system results in the risk of contaminants from the cell culture being present within the final product, including genomic materials from the host cells. Production of nucleic acids within cells is inefficient, since many more materials are required to be supplied to produce the nucleic acid than a synthetic method. In addition to the issues of cost, use of cell cultures can in many cases present difficulties for reproducibility of the amplification process. In the complex biochemical environment of the cell, it is difficult to control the quality and yields of the desired nucleic product. It is also difficult to deal with sequences that may be toxic to the cells in which the nucleic acid is amplified. Recombination events may also lead to problems in faithful production of a nucleic acid of interest.

DNA may be produced synthetically without the use of cells. Oligonucleotides may be synthesised chemically by extension of a chain using modified nucleotides. Preparation of these building blocks comes with a cost. The stepwise addition of each nucleotide is an imperfect process (the chance of each chain being extended is termed the ‘coupling efficiency’), and for longer sequences a majority of the initiated chains will not become full-length correct products. This precludes production of long sequences at large scale—there must always be a sacrifice between length, accuracy, and scale for these processes. Primary uses for such oligonucleotides are still in the low hundreds of nucleotide range (for example, primers and probes), and the maximum accurate length is thought to be around 300 nucleotides in length. Typically, synthetic oligonucleotides are single-stranded nucleic acid molecules around 15-25 bases in length.

A preferred alternative to synthetic processes is the enzymatic production of nucleic acids, which relies upon a template. Cell-free, in vitro enzymatic processes for the synthesis of nucleic acid avoids the requirement for use of any host cell, and so are advantageous, particularly when production is required to Good Manufacturing Practices (GMP) standards. Consequently, enzymatically produced nucleic acids can be made much more efficiently, and without the risk of cell-derived contaminants.

Therefore enzymatically produced and improved constructs which are safer and tolerable by the recipient are required, ideally that are also resistant to immediate degradation within the cell.

Making single stranded deoxyribose nucleic acid (DNA) vectors enzymatically can be problematic, since if a polymerase and primers are used with a double stranded template, inherently, production of two complementary strands occurs. Whilst these strands may be separated and the unwanted strand discarded, this can still be seen as a waste of processing resources. When scaling up production, the loss over 50% of starting materials in the final product is not sustainable.

The present invention relates particularly to a novel, cell-free and in vitro method for making single stranded nucleic acid constructs efficiently and effectively, and also to the templates that enable the production of the same. The templates enable the production of single stranded nucleic acid concatemers of any desirable length for various uses, including the production of single stranded nucleic acid constructs. These constructs are more stable than simple linear single stranded nucleic acids, due to the sequestering of the ends of the nucleic acid.

The available art does not disclose a process to manufacture single stranded nucleic acid with sequestered ends or a template for use in such a process as described herein.

Various documents describe the production of closed linear double stranded DNA with “capped” ends. WO2018/033730 of Touchlight IP relates to double stranded closed linear DNA molecules which would not be suitable for use as templates for the present invention, since there are no adjacent processing and conformational motifs. WO2019/051255 and WO2019/143885 of Generation Bio describes linear duplex DNA molecules formed from a continuous strand of complementary DNA with covalently-closed ends (linear, continuous and non-encapsidated structure), which comprise a 5′ inverted terminal repeat (ITR) sequence and a 3′ ITR sequence. Again, this would not be suitable for use as a template molecule according to the present invention.

Several RNA structures are already known, particularly in the field of CRIPSR-Cas 9 gene editing. In Gorter de Vries, et al. (Microb Cell Fact 16, 222 (2017). https://doi.org/10.1186/s12934-017-0835-1) discloses two ribozyme-flanked gRNAs which can self-process. A similar structure is detailed in Ng et al (Molecular Biology and Physiology, March/April 2017 Volume 2 Issue 2 e00385-16). Triple ribozyme (TRz) constructs which consist of two cis-acting ribozymes flanking an internal trans-acting ribozyme are disclosed in Benedict et al, Carcinogenesis. 1998 July; 19(7):1223-30. Such structures lack the adjacent conformational and processing motifs at both ends of the sequence of interest, allowing the sequestration of the terminal residue in the linear single stranded product.

### BRIEF DESCRIPTION OF FIGURES

FIG. 1 is an exemplary representation of a template of the present invention;

FIG. 2 is a representation of a different exemplary template and an expanded view of a single stranded nucleic acid produced by polymerase acting upon the template is shown;

FIG. 3 is a depiction of one method of amplification of a template of the invention, resulting in a nascent nucleic acid strand which is processed resulting in the single stranded nucleic acid construct with sequestered ends;

FIG. 4 provides two depictions of sections of the nascent strand from FIG. 3, with the processing step shown, resulting in the production of the single stranded nucleic acid construct with sequestered ends;

FIG. 5 shows an alternative representation of a template, together with the amplification and processing steps;

FIG. 6 is a gel photograph depicting the results of an assay of the results of Example 2, where nucleic acid constructs were tested for their resistance to exonuclease degradation;

FIG. 7 is a gel photograph depicting the results of an assay of the results of Example 3 where nucleic constructs were tested for their resistance to degradation by cellular components;

FIG. 8 is a representation of the sequence of an AAV2 ITR together with representations of possible conformations of single stranded nucleic acids of the invention with ITR-style structures;

FIG. 9 is a representation of the template used in Example 1; and

FIG. 10 is a gel photograph showing the results of Example 1.

### SUMMARY OF THE INVENTION

The single stranded nucleic acid molecule of the present invention has sequestered ends. The single stranded nucleic acid molecule of the present invention is a linear single strand of nucleic acid and therefore has a terminal nucleotide at each end. The terminal nucleic acid residues are not free, i.e. not exposed as in a purely linear single stranded nucleic acid molecule which has not assumed any further conformation. The ends of the nucleic acid are therefore secured or tucked away within the construct and are not immediately accessible to enzymes such as single strand nucleases and the like. The ends of the single stranded nucleic acid may be sequestered by including the terminal nucleotide within a conformation which acts to protect the ends. The terminal nucleotide at each end of the linear ssDNA is therefore kept apart or away from any agents which may act upon it in order to start to degrade the nucleic acid molecule. In general, enzymes locate the terminal nucleotides and from this residue start to chew up the single stranded nucleic acid.

The single stranded nucleic acid molecule may be prepared from a template nucleic acid. The design of this template nucleic acid is unique.

Accordingly, the present invention provides:

A nucleic acid template for the cell-free, in vitro manufacture of single stranded nucleic acid molecules with sequestered ends, comprising a sequence encoding the following elements:

i) a first processing motif, adjacent to

ii) a first conformational motif,

iii) a sequence of interest,

iv) a second conformational motif, adjacent to

v) a second processing motif,

wherein a processing motif includes sequences capable of forming a base-paired section including a recognition site for an endonuclease and an associated cleavage site, and wherein the conformational motif includes at least one sequence capable of forming intramolecular hydrogen bonds.

Thus, the template of the invention encodes the single stranded nucleic acid as described herein. The single stranded nucleic acid is linear. The linear single stranded nucleotide has sequestered ends.

Alternatively described, the combination of a processing motif and a conformational motif adjacent to each other in either the forward orientation (processing motif then conformational motif) or the reverse orientation (conformational motif then processing motif) can be used. These are the formatting elements.

Thus, the template may comprise the following sequences encoding the following elements in the order described:

i) a forward formatting element;

ii) a sequence of interest;

iii) a reverse formatting element.

The template of the invention may be amplified using any suitable polymerase enzyme, in order to manufacture the single stranded nucleic acid product.

The single stranded nucleic acid product is linear and has sequestered ends.

The template may be double or single stranded. One strand of the template is complementary to the desired linear single stranded nucleic acid product with sequestered ends, and therefore directs the production of the same. The template directs the construction of the product when contacted with a polymerase enzyme, and thus the template is replicated or amplified. The terms amplified or replicated may be used interchangeably in the art.

The template of the invention may be contacted with a polymerase capable of rolling circle amplification (RCA). The template of the invention may be amplified using a polymerase capable of catalysing rolling circle amplification (RCA). RCA is an isothermal enzymatic process where long single stranded DNA or RNA is synthesised using a circular DNA template and special DNA or RNA polymerases. The RCA product is a concatemer containing tens to hundreds or thousands of tandem repeats that are complementary to the circular template. Thus, the contacting of the template with a polymerase may result in an “amplification” of the template, producing a complementary single strand of nucleic acid.

Therefore, any template described herein may be amplified using a polymerase capable of rolling circle amplification or replication. This results in the production of a long single-stranded concatemeric nucleic acid molecule. Due to the presence of the formatting elements (comprising a processing motif adjacent to a conformational motif, in either the forward or reverse orientation), the concatemer can be simply processed by the addition of the requisite endonucleases. Cleavage within the processing motifs by the endonucleases releases the sequence of interest, flanked on either side by conformational motifs. As released, these conformational motifs act to sequester the ends of the single stranded nucleic acid by forming a hydrogen-bonded section which secures the terminal nucleotide. The conformational motif in the single stranded nucleic acid molecules do, therefore, assume a conformation using hydrogen bonding which sequesters the terminal nucleotide. The terminal nucleotide may be secured by being included within or embraced within the conformation assumed with or without intramolecular base-pairing or hydrogen bonding. Alternatively, the terminal nucleotide may be secured by intramolecular base-pairing or hydrogen bonding, such that the conformational motif increases the stability of these intramolecular interactions.

Thus, the terminal residues of the linear single stranded nucleic acid product are formed by the action of the endonuclease on the processing motif, the terminal residue is the residue at the end of the molecule once the endonuclease has cleaved the longer intermediate product. Thus, the formatting element may be described as comprising a processing motif adjacent to the conformational motif, wherein the cleavage site generates the terminal residue which is sequestered by the conformational motif. The processing motif and conformational motif can be described as adjacent, adjoining or contiguous. Alternatively described, there is no extraneous or intervening nucleic acid sequence between the processing motif and the conformational motif. The action of the endonuclease generates the terminal residue, which is subsequently sequestered.

Accordingly, the present invention provides:

A method of manufacturing single stranded nucleic acid molecules with sequestered ends, comprising:

(a) amplification of a circular template using a polymerase capable of rolling circle amplification, wherein said template comprises a sequence encoding the following elements:

i) a first processing motif, adjacent to

ii) a first conformational motif,

iii) a sequence of interest,

iv) a second conformational motif, adjacent to

v) a second processing motif,

wherein a processing motif includes sequences capable of forming a base-paired section including a recognition site for an endonuclease and an associated cleavage site, and wherein the conformational motif includes at least one sequence capable of forming intramolecular hydrogen bonds,

the amplification producing a nucleic acid concatemer, and

(b) processing said nucleic acid concatemer using one or more endonucleases which recognise the cleavage sites in one or more of said processing motifs.

The single stranded nucleic acid produced is linear, with sequestered ends.

Alternatively put, the invention comprises:

A method of manufacturing single stranded nucleic acid molecules with sequestered ends, comprising:

(a) the amplification of a circular template using a polymerase capable of rolling circle amplification, wherein said template comprises a sequence encoding the following elements:

i) a forward formatting element,

iii) a sequence of interest,

iv) a reverse formatting element,

wherein a forward formatting element comprises a processing motif adjacent to a conformational motif, and a reverse formatting element comprises a conformational motif adjacent to a processing motif; a processing motif includes sequences capable of forming a base-paired section including a recognition site for an endonuclease and an associated cleavage site, wherein the conformational motif includes at least one sequence capable of forming intramolecular hydrogen bonds,

the amplification producing a nucleic acid concatemer, and

(b) processing said nucleic acid concatemer using one or more endonucleases which recognise the cleavage sites in one or more of said processing motifs.

The single stranded nucleic acid molecules of the invention are linear, with sequestered ends.

The processing steps results in single stranded nucleic acid constructs with sequestered ends. The ends are sequestered since in the processed format, the conformational motifs are able to form or assume their desired conformation, which is stabilised by intramolecular hydrogen bonding. The end of the single stranded nucleic acid molecule is sequestered by the conformation assumed by the conformational motif. The terminal nucleotide may be secured by being included within a conformation, making it sterically difficult for it to be approached by exonucleases, or included in intramolecular bonding within the conformation motif, the entirety of which makes the terminal nucleotide more stable to exonucleases. Since the molecule has two ends and two conformational motifs, each works to assume a conformation embracing the relevant end or terminal nucleotide. The molecule has two ends, with two terminal residues, since the nucleic acid is linear.

The concatemer is an intermediate product during the manufacture of the single stranded nucleic acid molecules of the present invention, but may have some utility of its own, of its own due to its composition as a multimeric linked chain of sequences of interest which may serve to increase the local concentration or potency of said sequences in applications where that may be an advantage, for example in bio-sensing and the like. Affinity binding is one possible application.

Accordingly, the present invention provides:

A single stranded oligonucleotide concatemer with two or more repeats of a sequence unit, said sequence unit comprising the following elements:

i) a first processing motif, adjacent to

ii) a first conformational motif,

iii) a sequence of interest,

iv) a second conformational motif, adjacent to

v) a second processing motif,

wherein a processing motif includes sequences capable of forming a base-paired section including a recognition site for an endonuclease and an associated cleavage site, and wherein the conformational motif includes at least one sequence capable of forming intramolecular hydrogen bonds.

Alternatively put, the invention provides:

A single stranded nucleic acid concatemer with two or more repeats of a sequence unit, said sequence unit comprising the following elements:

i) a first processing motif, adjacent to

ii) a first conformational motif,

iii) a sequence of interest,

iv) a second conformational motif, adjacent to

v) a second processing motif,

wherein a processing motif includes sequences capable of forming a base-paired section including a recognition site for an endonuclease and an associated cleavage site, and wherein the conformational motif includes at least one sequence capable of forming intramolecular hydrogen bonds.

The single stranded nucleic acid molecules of the invention are linear, with sequestered ends.

Alternatively, if the processing motif and conformational motif are taken together as a processing element, the present invention provides:

A single stranded oligonucleotide concatemer with two or more repeats of a sequence unit, said sequence unit comprising the following elements:

i) a forward formatting element,

iii) a sequence of interest,

iv) a reverse formatting element,

wherein a forward formatting element comprises a processing motif adjacent to a conformational motif, and a reverse formatting element comprises a conformational motif adjacent to a processing motif; a processing motif includes sequences capable of forming a base-paired section including a recognition site for an endonuclease and an associated cleavage site, wherein the conformational motif includes at least one sequence capable of forming intramolecular hydrogen bonds.

The single stranded nucleic acid molecules of the invention are linear, with sequestered ends.

The terminal nucleotide of the conformational motif, or indeed the terminal nucleotide of the single stranded nucleic acid construct is usually the nucleotide which was adjacent to the processing motif and “released” from the concatemeric nucleic acid by the action of the endonuclease. It is this terminal nucleotide that forms the end of the single stranded nucleic acid construct, and is duly sequestered in order to delay degradation.

A forward formatting element comprises a processing motif adjacent to a conformational motif, and a reverse formatting element comprises a conformational motif adjacent to a processing motif. This arrangement ensures that a sequence of interest is flanked at each end by a conformational motif after processing. The sequence of interest is therefore flanked by two conformations in the construct, each sequestering an end of the nucleic acid.

### DETAILED DESCRIPTION OF THE FIGURES

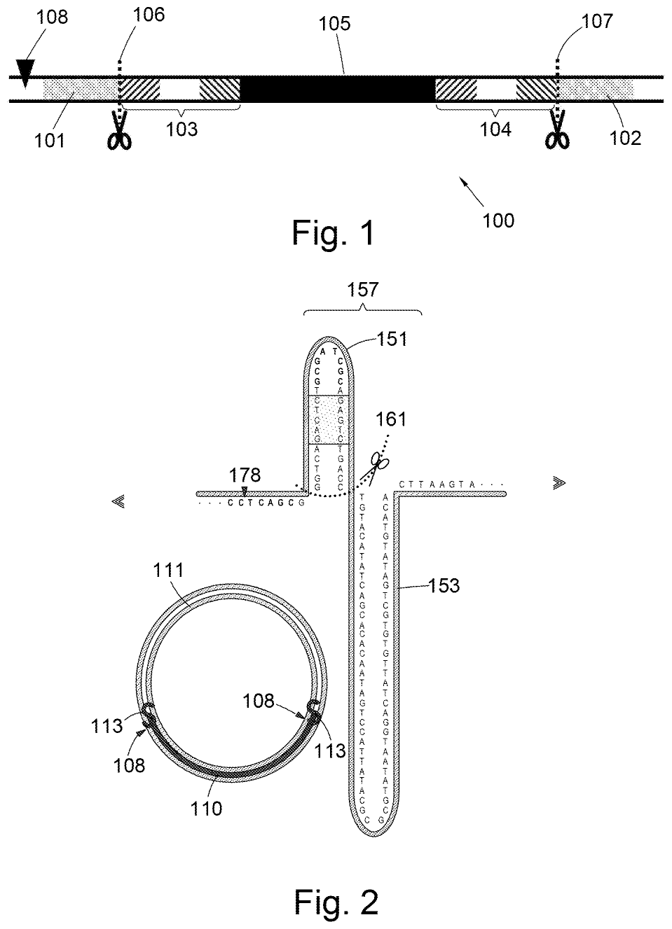

FIG. 1 is an exemplary representation of a template of the present invention (100). Shown are the sequences encoding the first and second processing motifs (101 and 102), the first and second conformational motifs (103 and 104), the sequence of interest (105), a recognition site for an endonuclease containing a cleavage site (106 and 107) and a nicking site in the template (108);

FIG. 2 is a representation of a different exemplary template (100), with the template depicted as a double stranded circular nucleic acid construct, with a nicking site (108) shown, together with the backbone sequence (110) and the sequence for production of the single stranded nucleic acid construct (111), terminating at each end with the sequence encoding the formatting element (113). An expanded view of a single stranded nucleic acid produced by polymerase amplification of a strand of the template at the formatting element (113) is shown. This depicts a nicking site (178), the first processing motif (151), the first conformational motif (153), and a recognition site for an endonuclease containing a cleavage site (161). The first processing motif and the first conformational motif are adjacent, separated only by the cleavage site, together these form the formatting element (157);

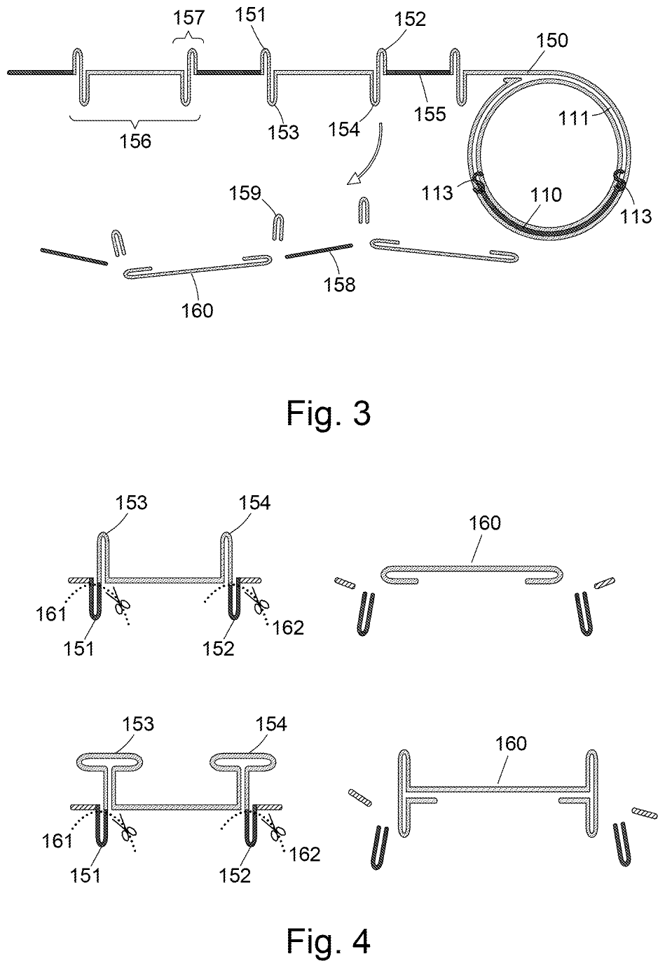

FIG. 3 is a depiction of one method of amplification of a template (100) of the invention, resulting in a nascent nucleic acid strand (150) using rolling circle amplification of the template. On the nascent strand, shown are the first processing motif (151), second processing motif (152), first conformational motif (153), second conformational motif (154), which together with the sequence of interest, form the basis for the nucleic acid construct (156), and the backbone sequence (155). The formatting element (157) is depicted, which includes the cleavage site (not shown). The nascent strand is processed using the requisite enzymes (not shown) which recognises the cleavage sites, resulting in the single stranded nucleic acid construct with sequestered ends (160), with the side products resulting from the template backbone (158) and processing motifs (159);

FIG. 4 provides two depictions of sections of the nascent strand from FIG. 3, with the processing step shown, resulting in the production of the single stranded nucleic acid construct with sequestered ends (160), with the side products. Depending on the type of conformational motif utilised, the conformation or structure of the 3′ and 5′ ends of the construct can vary;

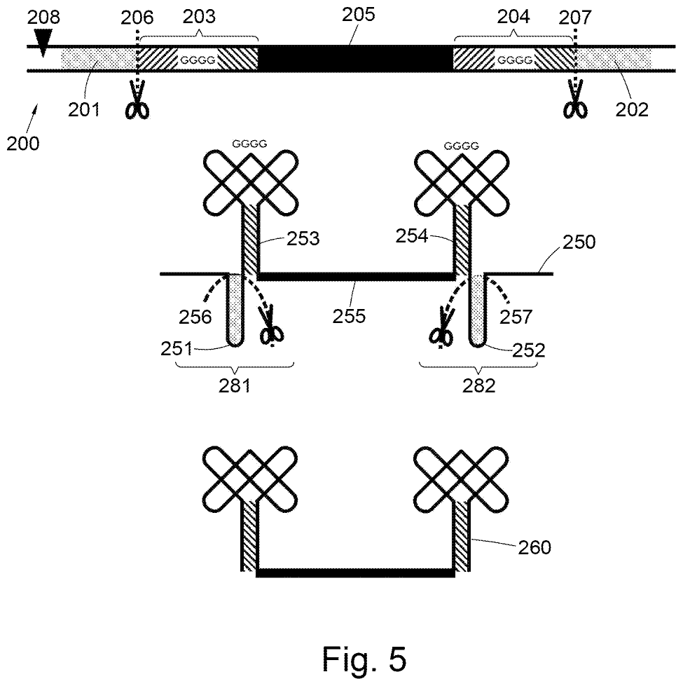

FIG. 5 shows an alternative representation of a template (200). Shown are the sequences encoding the first and second processing motifs (201 and 202), the first and second conformational motifs (203 and 204), the sequence of interest (205), a recognition site for an endonuclease containing a cleavage site (206 and 207) and a nicking site in the template (208). Also shown is the nascent strand of nucleic acid produced from the template (250). On the nascent strand there is shown the first processing motif (251), second processing motif (252), first conformational motif (253), and the second conformational motif (254), which together with the sequence of interest (255), form the nucleic acid construct. The cleavage sites (256 and 257) are formed within the formatting elements (281 and 282). The nascent strand is processed using the requisite enzymes which recognises the cleavage sites, resulting in the single stranded nucleic acid construct with sequestered ends (260), with the side products (not shown);

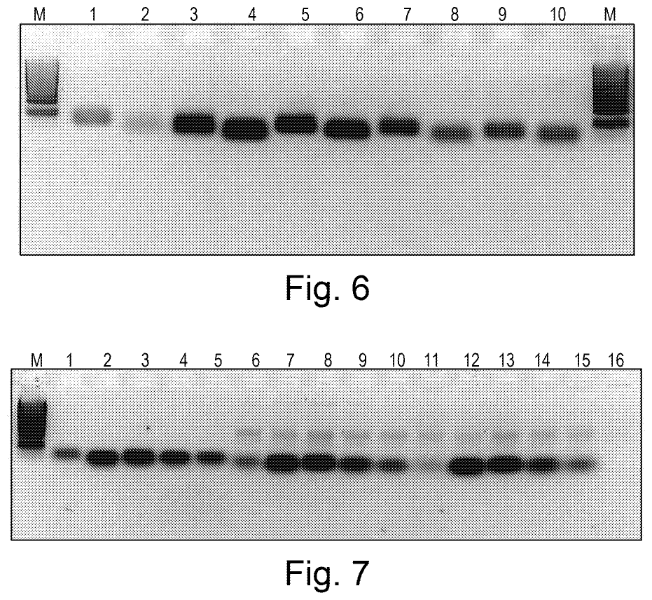

FIG. 6 is a gel photograph (0.8% TAE agarose gel, 1× GelRed stain) depicting the results of an assay of the results of Example 2. The lanes on the gel are as follow:

| M | ladder; 1 kb | |
|---|---|---|
| 1 | ssDNA (i) | no exo |
| 2 | ssDNA (i) | 100 U/mL exoVII |
| 3 | loops (GAA) (ii) | no exo |
| 4 | loops (GAA) (ii) | 100 U/mL exoVII |
| 5 | G-quadruplex (iii) | no exo |
| 6 | G-quadruplex (iii) | 100 U/mL exoVII |
| 7 | G-quadruplex (iv) | no exo |
| 8 | G-quadruplex (iv) | 100 U/mL exoVII |
| 9 | pseudoknot (v) | no exo |
| 10 | pseudoknot (v) | 100 U/mL exoVII |

Nucleic acid constructs are labelled in line with Example 2;

FIG. 7 is a gel photograph (0.8% TAE agarose gel, 1× GelRed stain) depicting the results of an assay of the results of Example 3. The lanes on the gel are as follow:

| M | ladder; NEB 1 kb | |
|---|---|---|
| 1 | ssDNA (i) | no extract added |
| 2 | loops (GAA) (ii) | no extract added |
| 3 | G-quadruplex (iii) | no extract added |
| 4 | G-quadruplex (iv) | no extract added |
| 5 | pseudoknot (v) | no extract added |
| 6 | ssDNA (i) | 5% extract, 24 h |
| 7 | loops (GAA) (ii) | 5% extract, 24 h |
| 8 | G-quadruplex (iii) | 5% extract, 24 h |
| 9 | G-quadruplex (iv) | 5% extract, 24 h |
| 10 | pseudoknot (v) | 5% extract, 24 h |
| 11 | ssDNA (i) | 5% extract, 72 h |
| 12 | loops (GAA) (ii) | 5% extract, 72 h |
| 13 | G-quadruplex (iii) | 5% extract, 72 h |
| 14 | G-quadruplex (iv) | 5% extract, 72 h |
| 15 | pseudoknot (v) | 5% extract, 72 h |
| 16 | 5% cell extract; no DNA added | |

Nucleic acid constructs are labelled in line with Example 2;

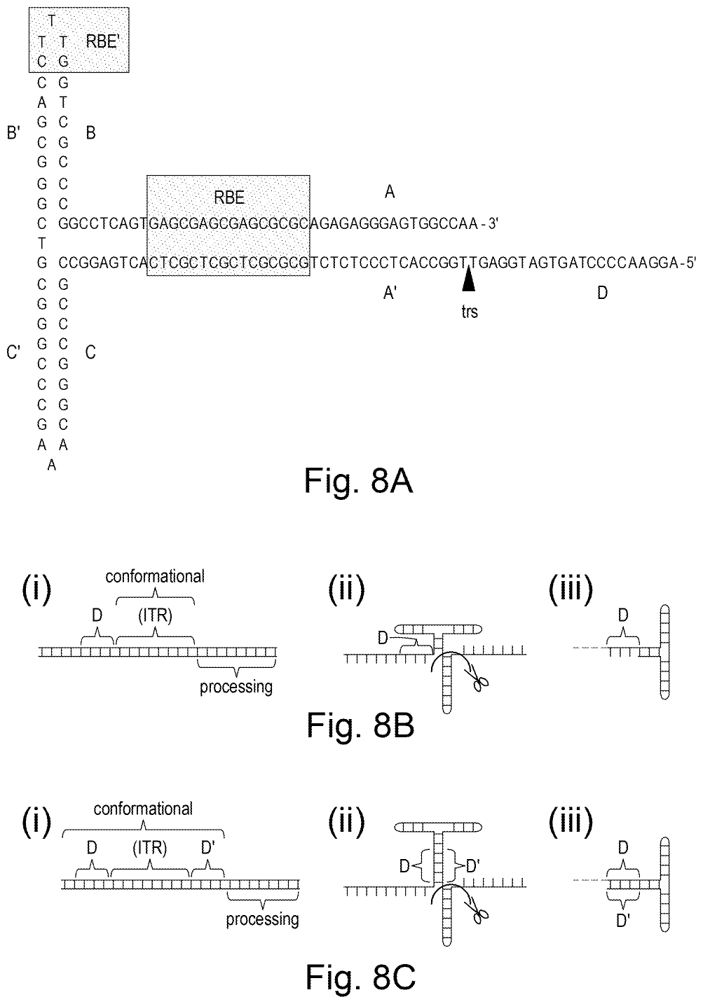

FIG. 8A is a representation of the conformation of the AAV2 ITR. The AAV2 ITR is composed of two arm palindromes (B-B′ and C-C′) embedded in a larger stem palindrome (A-A′). The ITR can acquire two configurations (flip and flop). The flip (depicted) and flop configurations have respectively the B-B′ and the C-C′ palindromes closest to the 3′ end. The D sequence is present only once at each end of the genome thus remaining single-stranded. The boxed motif corresponds to the Rep-binding element (RBE). FIGS. 8B and 8C are representations of the template for a linear single stranded nucleic acid of the invention (i), followed by the single stranded product of the template both before (ii) and after (iii) cleavage. FIG. 8B includes a single stranded D region as in the wild type AAV ITR. FIG. 8C includes the D region within the conformational motif by pairing it with a D′ region, and it is thus in a double stranded portion;

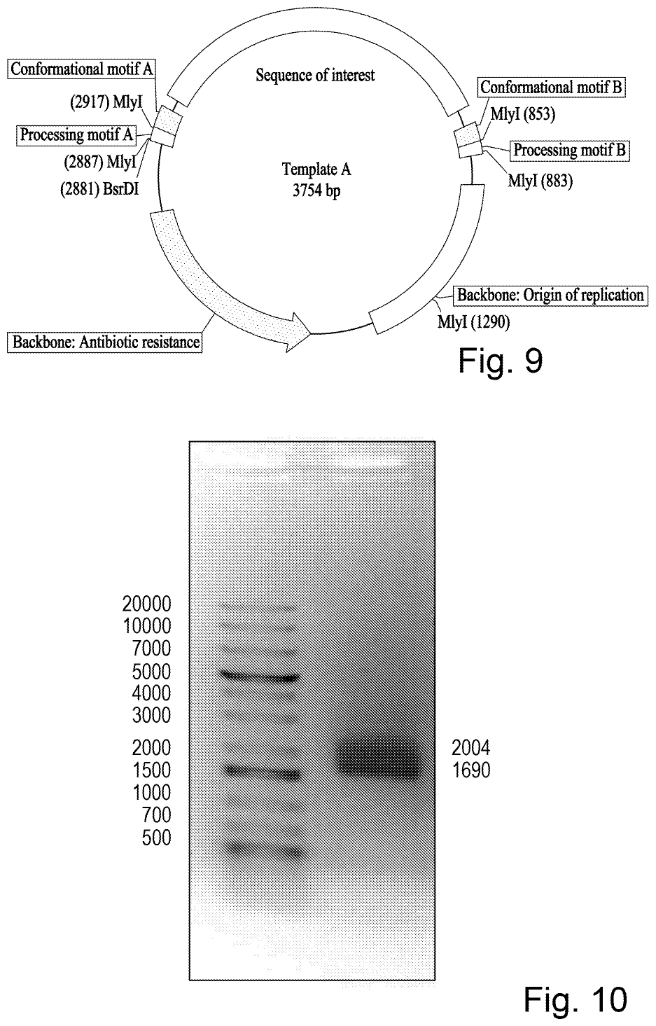

FIG. 9 is a representation of a template used in Example 1, a plasmid map. Shown are: the sequence of interest, conformational motifs, processing motifs, backbone and sites for the processing enzyme (MlyI). A site for a nicking endonuclease (BsrDI, which may be nicked by a variant of Nb.BsrDI for example) is also depicted; and

FIG. 10 is a gel photograph (0.8% TAE agarose gel, 1× SafeView stain) with two lanes shown, the first lane is a marker lane, and the second shows the nucleic acid construct made using the template of FIG. 9 according to Example 1.

### DETAILED DESCRIPTION OF THE INVENTION

The present invention meets the need of an efficient, cell-free, enzymatic, cost-effective, accurate and clean method of manufacturing large-scale amounts of a single stranded nucleic acid molecule in vitro. In order to increase the longevity of the single stranded nucleic acid molecule for cell-based uses, the present inventors have devised an elegant way of protecting the ends of the single stranded nucleic acid molecule from immediate degradation by sequestering these ends.

Sequestered End

A key feature of all linear nucleic acid molecules is that they are a polymer comprising nucleotide residues and have two distinctive ends. The nature of the ends is dictated by the nature of the backbone for the nucleic acid. For natural (non-synthetic) nucleic acid molecules these two ends are the 5′ (5-prime) and 3′ (3-prime) ends. In natural nucleic acids (i.e. DNA or RNA), the 5′ end is that end of the molecule which terminates in a 5′ phosphate group. By convention, nucleic acid sequences are written with the 5′ end to the left and the 3′ end to the right, and the orders recited herein are in line with that convention. The 3′ end is that end of the molecule which terminates in a 3′ phosphate group. Generally in natural nucleic acids, a phosphodiester linkage forms between the phosphate group of one nucleotide and the sugar of another nucleotide to form the backbone. Using the chemical convention for carbon numbering in nucleotides, the phosphate group is the 5′ end of a nucleotide because it is bonded to the 5′ carbon of the sugar. Phosphodiester linkages form between the 5′ end of one nucleotide and the 3′ hydroxyl group of another nucleotide, forming a polymer with one open 5′ end and one open 3′ end. The 5′ end may therefore be considered to be the terminal residue with a 5′ phosphate group. The 3′ end may therefore be considered to be the terminal residue with a 3′ hydroxyl group. For DNA and RNA, these terminal residues are nucleotide residues.

In the present invention, the ends of the linear single stranded nucleotide are formed by the action of endonucleases on the intermediate product of the method of the invention. Thus, the terminal residue of the conformational motif becomes the terminal residue of the single stranded nucleic acid product. Prior to cleavage, this residue effectively connected the conformational motif to the processing motif.

Nucleic acids can only be synthesized in vivo in the 5′-to-3′ direction, as the polymerases that assemble new strands commonly rely on the energy produced by breaking nucleoside triphosphate bonds to attach new nucleoside monophosphates to the 3′-hydroxyl (—OH) group, via a phosphodiester bond. The relative positions of entities along a strand of nucleic acid, including genes and various protein binding sites, are commonly noted as being either upstream (towards the 5′-end) or downstream (towards the 3′-end). In nature, due to the anti-parallel nature of DNA, this means the 3′ end of the template strand is upstream of a gene and the 5′ end is downstream.

For non-natural (synthetic) nucleic acids which are entirely synthetic the ends may be labelled according to the backbone structure. For example, if peptide nucleic acid (PNA) is examined, the sugar phosphate backbone has been replaced by a unit of N-(2 aminoethyl) glycine. Each of the 4 natural bases is then connected to the backbone via a methylene carbonyl linker. PNA has an N-terminal end and a C-terminal end, rather than 5′ and 3′ ends.

In the present invention, the ends of the linear nucleic acid molecule are sequestered, no matter the nomenclature of these ends. Accordingly, the terminal residues or terminal nucleotides at these ends are not free or exposed. For natural nucleic acids, such as DNA and RNA, these terminal residues are terminal nucleotides, and are the 3′ and 5′ ends. For synthetic nucleic acids, these ends may have their appropriate nomenclature.

Each sequestered end is stabilised, such that it is no longer available for immediate reaction with enzymes such as single strand nucleases. If the nucleic acid is for use in a cellular environment, the end is kept away, shielded or secluded from the cellular components that may cause immediate degradation of the single stranded nucleic acid. Therefore, the ends of the single stranded nucleic acid molecule do not act as they would do normally, in the absence of sequestration. The sequestration of the ends affords the molecule an enhanced stability compared to analogous molecules without sequestered ends. This is demonstrated by the Inventors in Example 1, wherein an analogous molecule without sequestered ends is degraded, whereas the molecule of the invention remains intact.

It is preferred that the end is sequestered by the presence of the conformational motif. The conformational motif has a particular sequence. The sequence of the conformational motif is designed such that it is capable of forming intramolecular hydrogen bonds in order to form or assume a particular conformation. When the conformation is assumed in the single stranded nucleic acid construct, the terminal nucleotide is sequestered by the motif, which means that is has been secured.

The intramolecular hydrogen bonds may be within the conformational motif sequence itself, or may be between a portion or part of the conformational motif and at least one other sequence in the whole single stranded nucleic acid molecule, such as the sequence of interest. The intramolecular hydrogen bonds may or may not include the terminal nucleotide.

Hydrogen bonding is a non-covalent type of bonding between molecules or within them, intermolecularly or intramolecularly. These bonds are formed from an electronegative atom (the hydrogen acceptor) and a hydrogen atom that attaches covalently with another electronegative atom (the hydrogen donor—only nitrogen, oxygen, and fluorine atoms will work) of the same molecule or of a different molecule. They are the strongest kind of dipole-dipole interaction. Hydrogen bonds are responsible for specific base-pair formation in a DNA double helix and are a factor to the stability of a DNA double helix structure.

Typically, in Watson-Crick base-pairing, hydrogen bonds form between the nitrogenous bases of the nucleotides (nucleobases). In standard base pairings, which are adenine-thymine (A-T) in DNA, adenine-uracil (A-U) in RNA and cytosine-guanine (C-G) in both, hydrogen bonds form. The A-T/U and C-G pairings function to form double or triple hydrogen bonds between the amine and carbonyl groups on the complementary bases.

A wobble base pair is a pairing between two nucleotides in nucleic molecules, most notably in RNA, that does not follow standard Watson-Crick base pair rules. The four main wobble base pairs are guanine-uracil (G-U), hypoxanthine-uracil (I-U), hypoxanthine-adenine (I-A), and hypoxanthine-cytosine (I-C). The thermodynamic stability of a wobble base pair is comparable to that of a Watson-Crick base pair. Wobble base pairs are fundamental in RNA structure.

Alternative or non-canonical base-pairings are also possible in nucleic acid structures, again held together by hydrogen bonds. These are generally more common in RNA, but are also possible in DNA and other nucleic acids. One example of non-canonical base pairing is Hoogsteen and reverse Hoogsteen base-pairing. In these interactions, the purine bases, adenine and guanine, flip their normal orientation and form a new set of hydrogen bonds with their partners. Hoogsteen hydrogen bonding has been shown to be present in quadruplexes such as the i-motif and G-quadruplex discussed in more detail herein.

A combination of various base-pairing mechanisms can also be envisaged. For example, when the hydrogen bonds in the A-T and G-C base pairs in canonical B-form DNA are formed, several hydrogen bond donor and acceptor groups in nucleobases remain unused. Each purine base has two such groups on the edges that are exposed in the major groove. Triplex DNA may form intermolecularly, between a duplex and a third oligonucleotide strand. The third strand bases may form Hoogsteen-type hydrogen bonds with purines in the B-form duplex.

Base-pairs may also form between natural and non-natural bases, and also between pairs of non-natural bases.

Therefore, base-pairing is an example of the intramolecular hydrogen bonding enabling the conformational motif to assume the relevant conformation. If the conformational motif relies upon base pairing to sequester the terminal nucleotide, there may be a sequence within said motif that base-pairs to a sequence elsewhere in the single stranded nucleic acid construct (i.e. within the sequence of interest). Alternatively, a sequence within the conformational motif may be designed to base-pair with at least one other sequence within the conformational motif, such that the hydrogen bonds are formed within the motif itself. Any type of base-pair is envisaged, including those that form between nucleotides that are “non-complementary” according to standard Watson-Crick pairing.

The intramolecular hydrogen bonds may also be interactions which are not defined as classical base pairing, such as the planar arrangement of guanine residues in the G-tetrad of a G-quadruplex, which is stabilised by Hoogsteen hydrogen bonding. These structures are discussed further below.

Further, stabilisation of nucleic acid molecules may also rely upon base-stacking interactions. Pi-pi stacking (also called π-π stacking) refers to attractive, noncovalent interactions between aromatic rings, since they contain pi bonds. These interactions are important in nucleobase stacking within nucleic acid molecules, which have been brought together by hydrogen bonding. It is thus likely that the single stranded nucleic acid constructs are further stabilised by base-stacking interactions. Other interactions stabilising the nucleic acid are also possible, these include pi-cation interactions, Van der Waals interactions and hydrophobic interactions.

In one aspect, the conformational motif is designed to include a sequence to enable a base paired section to form. The base paired section may include an appropriate number of nucleotides in the base-paired section. In some aspects the base-paired section may be formed of a sequence of nucleotides. Due to the need to maintain a conformation, the base paired section is likely to be at least 5 base pairs in length. The base paired section may include at least 2 nucleotides, or 2-5 nucleotides, or 5 nucleotides, or may include 5 or more nucleotides, i.e. 5, 6, 7, 8, 9, 10, 11, 12, 13, 14, 15 or more nucleotides. In some instances the base paired section may include many more nucleotides in order to securely sequester the terminal nucleotide. Therefore, the base paired section can be 1-50 or 1-100 nucleotides in length, or indeed 1-250 nucleotides or more.

The terminal nucleotide residue may be hydrogen-bonded intramolecularly to another part of the single stranded nucleic acid construct, including the conformational motif. In one aspect, the terminal nucleotide forms a base-pair with another nucleotide in the construct.

The terminal residue may, however, be free from hydrogen bonding or more particularly base-pairing. In this instance, the conformational motif secures or sequesters the terminal nucleotide by embracing, encircling or surrounding the terminal nucleotide, such that it is not free for a single strand nuclease to cleave it from the adjacent nucleotide in the construct (and then cleave the adjacent nucleotide and so on). In other words, the end is sterically protected from degradation, as it is not possible for larger entities to reach it. As an example, terminal nucleotides may be secured within a quadruplex motif.

It may simply be that it is the terminal residue at each end of the single stranded nucleic acid molecule that is sequestered. Alternatively, the adjacent one or more residues may also be sequestered. At least 5 or more, 10 or more, 15 or more, 20 or more, 25 or more, 50 or more residues may also be sequestered along with the terminal residue.

In a further aspect, each end may be sequestered by the formation of a duplex including at least the terminal residue at the end of the molecule. The duplex is formed by base-pairing between nucleotide sequences. These sequences may be adjacent (hairpin) or separated (stem loop etc.).

A residue refers to a single unit that makes up a nucleic acid polymer, such as a nucleotide.

In a further aspect, it is preferred that the base-paired or duplex section which acts to sequester the end or terminal nucleotide of the single stranded nucleic acid construct forms within the conformational motif. Thus, the conformational motif includes self-complementary sequences that are capable of forming a base-paired or duplex section. These may be adjacent or separated by non-complementary sequences.

In other aspects, the base paired or duplex section which acts to sequester the end or terminal nucleotide of the single stranded nucleic acid construct forms outside of the conformational motif. Thus, it may involve part of the sequence of interest, or indeed a spacer sequence that could be introduced within the nucleic acid construct (i.e. between 2 coding regions in the “sequence of interest”). The conformation achieved may thus be a lariat, which is a loop of single stranded nucleic acid which comprises a section of annealed complementary sequence or duplex comprising the terminal residue.

In some interesting aspects discussed further herein, the end may be sequestered within conformations such as quadruplexes. These are quadruple (four stranded) structures, which may be involved in the structure of telomere ends of chromosomes. The underlying pattern is a tetrad, a planar arrangement of 4 residues, stabilised by Hoogsteen hydrogen bonding and coordination to a central cation. A quadruplex is formed by stacking of multiple tetrads. Many different topologies may form depending upon how the sequence initially folds into these arrangements. The quadruplex structure may be further stabilized by the presence of a cation, especially potassium, which sits in a central channel between each pair of tetrads. Quadruplexes have been shown to be possible in DNA, RNA, LNA, and PNA, and may be intramolecular.

Exemplary quadruplexes include G-quadruplexes, which are formed from G-rich sequences and i-motifs (intercalated motif) formed by cytosine-rich sequences.

In one aspect, therefore, the terminal nucleotide is sequestered within a quadruplex, optionally a G-quadruplex or an i-motif.

Conformational Motif

One of the desired products is a single stranded nucleic acid molecule or construct, composed of any suitable nucleic acid, but preferably DNA or RNA, which contains a sequence of interest flanked on both sides by conformational motifs that sequester the ends of the single strand. The single stranded nucleic acid construct therefore has a first (generally at the 5′ end) and a second (generally at the 3′ end) conformational motif. Each conformational motif can be unique, but they all share the property that they are capable of sequestering the end of the single strand.

The single stranded nucleic acid molecule or construct may include any suitable conformational motif, as discussed in related to the sequestered ends.

The conformational motif comprises a sequence that is capable of forming intramolecular hydrogen bonds. These hydrogen bonds may be base pairs of any kind, or Hoogsteen type hydrogen bonds seen in structures such as tetraplexes/quadruplexes.

Notably, a conformational motif may be a sequence that includes one or more sections of sequence that are capable of forming base-pairs to another section of sequence either within the conformational motif itself, elsewhere within the single stranded nucleic acid.

The conformational motif may therefore simply include two sections of sequence that are “complementary” and that base-pair to form an antiparallel or indeed parallel duplex. This duplex may or may not include the terminal residue (i.e. 3′ or 5′ end) of the single stranded nucleic acid. In this instance, the conformational motif may form a hairpin (the two sections are contiguous) or stem loop (if the two sections are separated by a spacer sequence leaving single stranded nucleic acid). It will be understood that such a structure may be achieved by including an inverted repeat sequence in the conformational motif. A palindromic sequence is a section of double stranded nucleic acid sequence wherein reading 5′ to 3′ forward on one section matches the sequence reading 5′ to 3′ forward on the complementary section with which it forms a duplex.

The conformational motif may therefore include sequences necessary for the formation of one or more of: hairpins, stem loops, or pseudoknots. All of these conformations have in common two sections of sequence which can form a duplex. Alternative structures include lariats or lassos, which also include sections of sequence which can form a duplex.

The conformational motif can be a hybrid of different conformations, such as a G quadruplex with an additional sequence designed to form a duplex, in order to sequester the end by direct base-pairing. All that is necessary is that the conformational motif can secure the terminal nucleotide.

Organisms with single stranded DNA or RNA genomes, or organisms where genetic material may exist as a single strand for part of the life cycle, have evolved to protect the free ends of the nucleic acid by using particular structures, or by other means, including the positioning of proteins. Indeed, mammalian genomes have evolved the use of telomeres to protect the end of chromosomes where there may be a single strand overhang.

For Example, AAV protects the ends of the single stranded DNA genome using ITRs. Adeno-associated virus (AAV) is a nonpathogenic member of the Parvoviridae family. The wild-type AAV genome contains inverted terminal repeats (ITRs) that usually consist of 145 nucleotides at both ends. The terminal 125 nucleotides of each ITR may self-anneal to form a palindromic double-stranded T-shaped hairpin structure, in which the small palindromic B-B′ and C-C′ regions form the cross arm and the large palindromic A-A′ region forms the stem. Each structure is followed by a unique approximately 20-nucleotide D (or D′) region. Recombinant AAV (rAAV) production may not be affected by truncations within the ITRs, resulting in lengths of 137 nucleotides or less. In nature, the ITR serves as origin of replication and is composed of two arm palindromes (FIG. 8A—B-B′ and C-C′) embedded in a larger stem palindrome (A-A′). The ITR can acquire two configurations (flip and flop). The flip (depicted in FIG. 8A—AAV2) and flop configurations have respectively the B-B′ and the C-C′ palindromes closest to the 3′ end. The boxed motif corresponds to the Rep-binding element (RBE) where the AAV Rep proteins bind. The RBE may consist of a tetranucleotide repeat with the consensus sequence 5′-GNGC-3′.

Previously it has been shown (Ping et al, Mol Biotechnol DOI 10.1007/s12033-014-9832-3) that the presence of the D region in single stranded DNA (as shown in FIG. 8B) may be deleterious to expression of the transgene carried by the recombinant AAV vector. It is thought that the D region provides a binding site for a human protein that preferentially binds to this region and prevents second strand synthesis (Qing et al, Proc. Natl. Acad. Sci. USA, Vol. 94, pp. 10879-10884, September 1997, Medical Sciences and Kwon et al, Human Gene Therapy, DOI: 10.1089/hum.2020.018). Therefore, it may not be desirable to include a D region in single stranded DNA of the invention if it is the intention to express a transgene within a cell, and this can be done by removing the sequence or providing it within the conformational motif, such that it is paired with a D′ region as shown in FIG. 8C. Thus, not only is there a D region present, but also a paired D′ region. Such a pairing may offer additional stabilisation of ITR-style structures. Further, the presence of a double stranded D region may permit transcription factors (such as RFX) to bind, and potentially also enhance nuclear transport (Julien et al, Sci Rep. 2018 Jan. 9; 8(1):210. doi: 10.1038/s41598-017-18604-3.) An additional advantage of the presence of a D region may be the potential for the presence of this region to dampen host humoral immune response. Kwon et al have shown that the D sequence may inhibit expression of MHC-II genes. Thus, if the D region is present, but in double stranded form, it has the advantage of dampening the host immune response and avoiding the inhibition of second strand synthesis seen when the D region is in single stranded form, whilst potentially providing a mechanism for nuclear transport. The presence of a double stranded D region within the nucleic acid construct, particularly in the conformational motif, is therefore desirable.

Thus the invention extends to a linear single stranded nucleic acid molecule with sequestered ends, wherein at least one end comprises an ITR structure including a double stranded D region. Said D region may be in a duplex with a D′ region. As used herein a D′ region is sufficiently complementary to a D region to allow a duplex to form between the two sequences. The D region may be a natural D region sequence (in FIG. 8A—AGGAACCCCTAGTGATGGAG, SEQ ID No. 2, various serotype variants presented as SEQ ID No. 3 to 6) or a sequence with sufficient homology thereto, such as at least 80, 85, 90, 95 or 99% homology. The linear single stranded DNA of the invention may include one or two ITR ends as described here. These ends may be the same or different. The advantage of such a structure is that any transgene would be expressed whilst the host immune system may be temporarily suppressed.

The conformational motif of the single stranded nucleic acid construct may therefore be an ITR sequence taken from any AAV serotype. It may be a derivatised sequence based on an ITR from any AAV serotype, for example one or more of the elements may be amended, altered or replaced. The RBE can be removed, or the length of either palindrome can be modified, depending on the use to which the single stranded nucleic acid construct will be put. The conformational motif can be an entirely different sequence to natural AAV ITR sequences but still maintain a similar structure. Those skilled in the art would appreciate how to design a sequence that would form a two armed palindrome, using appropriate self-complementary sequences.

Other viral genomes also rely upon sequestered ends at the end of their linear genomes. HIV has at least a 5′ sequestered end.

Alternatively, the use of folding structures such as G-quadruplexes and intercalated motifs (i-motifs), may be considered. i-motifs and G-quadruplexes are four-stranded quadruplex structures formed by DNA; i-motifs are formed by cytosine-rich DNA regions, and G-quadruplexes by guanine-rich DNA forms. I-motifs have potential applications in nanotechnology and nanomedicine due to being particularly stable at pH values below physiological, and have been used as biosensors, nanomachines, and molecular switches.

The sequences of G-quadruplexes are varied and may be defined by the putative formula: (G₃₊N₁₋ₙG₃₊N₁₋ₙG₃₊N₁₋ₙG₃₊) where N is any nucleotide, including guanine. The number of residues between the Guanines defines the lengths of the loops. Loops larger than 7 nucleotides have been seen.

The conformational motif therefore assumes a conformation held by hydrogen bonding that may be further stabilised by interactions such as base-stacking. These conformations may indeed be further stabilised by the presence of small molecules or ions, examples of which are given below.

Quadruplexes (alternatively called tetraplexes) may complex around a central ion, for example. A number of ligands, both small molecules and proteins, can bind to quadruplexes. These ligands can be naturally occurring or synthetic. It has been found that all characterized G-quadruplex binding proteins share a 20 amino acid long motif/domain (RGRGR GRGGG SGGSG GRGRG—SEQ ID No. 7) called NIQI (Novel Interesting Quadruplex Interaction Motif) which is similar to the previously described RG-rich domain (RRGDG RRRGG GGRGQ GGRGR GGGFKG—SEQ ID No. 8) of the FMR1 G-quadruplex binding protein. Cationic porphyrins have been shown to bind intercalatively with G-quadruplexes. It may be important to match the quadruplex which has stacked quartets and the loops of nucleic acids holding it together. π-π interactions may be important determiners for ligand binding. Ligands should have a higher affinity for parallel folded quadruplexes. Ligands that bind to other conformational motifs to stabilise them are also contemplated.

The conformational motif sequesters the end of the single stranded nucleic acid molecule, and generally forms a particular structure. The conformational motif may be designed such that this structure has its own function, further to sequestering the end. For example, it can be designed such that an aptamer is formed by the conformational motif, or ribozymes, deoxyribozymes, and riboswitches. Aptamers bind to specific targets because of electrostatic interactions, hydrophobic interactions, and their complementary shapes. It is possible to engineer aptamer sequences through repeated rounds of in vitro selection or SELEX (systematic evolution of ligands by exponential enrichment) to bind to various molecular targets such as small molecules, proteins, nucleic acids, and even to larger entities such as cells, tissues and organisms. Alternatively, the conformational motif can be designed to include sequences that facilitate crossing the cell or nuclear membranes. Additionally or alternatively, the conformational motif may be designed to allow for formation of oligomeric complexes using the nucleic acid constructs, which may be of use in nanotechnology and the like.

Nucleic acid conformations can be affected by changes in conditions. The sequences for the conformational motif should be selected such that the conformation is adopted under the conditions under which the nucleic acid construct is to be used (i.e. pH, temperature, salt concentration, pressure, protein concentration, sugar concentration, osmotic pressure and the like). The nucleic acid construct can be used in many various conditions, such as physiological conditions or conditions that favour use of the technology in electronics for example.

Physiological conditions are conditions of the external or internal milieu that may occur in nature for that organism or cell system, and may be the appropriate conditions for the conformational motif to assume the relevant conformation.

Should the nucleic acid construct be used for non-cellular purposes, i.e. in nanotechnology, the conformation may be achieved in the relevant buffer solution, or indeed in pure water, as required.

Thus, the conformational motif can be in single stranded format in the concatemeric precursor molecule, these may be conditions under which no conformation is assumed, or indeed are possible. In the concatemeric precursor it will be understood that the terminal residue is contiguous with the processing motif. It is the adjacent nature of the motifs that allows for the production of linear single stranded nucleic acid molecules with sequestered ends.

Sequence of Interest

The single stranded nucleic acid construct also comprises a sequence of interest. It will be understood that the sequence of interest may contain more than one sequence, and indeed may contain many sequences, for example several gene sequences may be included within the “sequence of interest”, each of which may have associated promoters and enhancer elements, if required.

The sequence of interest may also include spacer sequences which include sequences with complementarity to the sequence of the conformational motif, to enable a base paired section to form to sequester the end or terminal nucleotide.

This sequence of interest may be any suitable sequence, or include any number of sequences. The sequence may itself have a function, such as forming an aptamer, a nucleic acid enzyme, ribozymes, deoxyribozymes, riboswitches, small interfering RNA, or the like. The sequence of interest may encode a product, which may be an aptamer, a protein, a peptide, or RNA, such as small interfering RNA. The sequence of interest may include an expression cassette comprising one or more promoter or enhancer elements and a gene or other coding sequence which encodes an mRNA or protein of interest. The expression cassette may comprise a eukaryotic promoter operably linked to a sequence encoding a protein of interest, and optionally an enhancer and/or a eukaryotic transcription termination sequence.

Alternatively, the sequence of interest may be designed to be a carrier sequence. Thus, the sequence of interest may be sufficiently complementary to another separate sequence which may anneal to it, such that the entire single stranded nucleic acid carrier is effectively used as a delivery mechanism for another molecule, by forming a duplex with the single stranded section. The separate oligonucleotide may be entirely synthetic. In this context, the single stranded product acts as a “carrier” molecule.

The sequence of interest may be used for production of DNA for expression in a host cell, particularly for production of DNA vaccines. DNA vaccines typically encode a modified form of an infectious organism's DNA. DNA vaccines are administered to a subject where they then express the selected protein of the infectious organism, initiating an immune response against that protein which is typically protective. DNA vaccines may also encode a tumour antigen in a cancer immunotherapy approach.

The sequence of interest may produce other types of therapeutic DNA molecules e.g. those used in gene therapy. For example, such DNA molecules can be used to express a functional gene where a subject has a genetic disorder caused by a dysfunctional version of that gene. Examples of such diseases are well known in the art.

The sequence of interest may be capable of acting as donor nucleic acid for gene editing purposes, both in animals and plants. Exemplary methods of gene editing include CRISPR gene editing and Transcription activator-like effector nucleases (TALENs) based methods.

The novel structures of the invention may also have non-medical uses including in material science, in nanotechnology, data storage and the like, and the sequence of interest can be selected accordingly. The nucleic acid may be used in bio-batteries, security marking of objects, or as biomolecular electronic components.

It is preferred for therapeutic uses in particular that the single stranded nucleic acid construct with sequestered ends lacks a bacterial origin of replication, lacks resistance genes (i.e. for antibiotics), lacks CpG islands (except for DNA vaccines where the same may be helpful), lacks methylation of cytosine and adenine, and is devoid of sequences that would identify the nucleic acid as foreign to the host cell (if the construct is for cellular uses).

The single stranded nucleic acid construct may be a natural nucleic acid molecule such as DNA or RNA. It is preferred that the single stranded nucleic acid construct is DNA. The single stranded nucleic acid construct can also be a non-natural nucleic acid molecule. Examples of non-natural nucleic acid molecules or xeno nucleic acids (XNA) include 1,5-anhydrohexitol nucleic acid (HNA), cyclohexene nucleic acid (CeNA), threose nucleic acid (TNA), glycol nucleic acid (GNA), locked nucleic acid (LNA), peptide nucleic acid (PNA) and FANA. Hachimoji DNA is a synthetic nucleic acid analogue that uses four synthetic nucleotides in addition to the four/five present in the natural nucleic acids, DNA and RNA. Enzymes have been engineered, mutated or developed in order to recognise synthetic nucleic acid molecules, and therefore the methods and products of the invention apply equally to these analogues, or hybrids of synthetic and natural nucleic acids and chimeras thereof.

Making the Single Stranded Nucleic Acid Molecule/Construct

The single stranded nucleic acid construct may be made using a unique method by rolling circle amplification of the distinctive templates, and then processing the single stranded nucleic acid concatemer that results from this amplification.

The method of manufacturing the single stranded nucleic acid construct with sequestered ends relies upon the amplification of a template nucleic acid (a “sequence unit”) by rolling circle amplification with a relevant polymerase enzyme, resulting in the production of a long, single stranded nucleic acid with multiple repeats of the sequence unit encoded by the template. This concatemeric single stranded nucleic acid may then then processed into the product, single stranded nucleic acid with sequestered ends.

The amplification process will require the addition of substrates (i.e. appropriate nucleosides for nucleic acid generation), and any co-factors (such as salts, ions or the like). Appropriate conditions including the presence of buffers and temperatures at which the enzymes can operate. Appropriate conditions for rolling circle amplification may be isothermal.

Amplification is the production of multiple copies of a nucleic acid template, or the production of multiple nucleic acid sequence copies that are complementary to the nucleic acid template. In the methods of the invention, it is preferred that amplification refers to the production of multiple nucleic acid sequence copies that are complementary to the nucleic acid template.

It is preferred, where the template is double stranded, that techniques are used to ensure that the strand complementary to the desired product is used as the template. This may be achieved by several methods discussed further below.

When used, nucleosides are compounds wherein a nucleic acid base (nucleobase) is linked to a sugar moiety. The nucleic acid base may be a natural or a modified/synthetic nucleobase. The nucleic acid base may include a purine base (e.g., adenine or guanine), a pyrimidine (e.g., cytosine, uracil, or thymine), or a deazapurine base, amongst others. The nucleic acid base may be a ribose or a deoxyribose sugar moiety. The sugar moiety may include a natural sugar, a sugar substitute, a substituted sugar, or a modified sugar. The nucleoside may contain a 2′-hydroxyl, 2′-deoxy, or 2, 3′-dideoxy forms of the sugar moiety.

Nucleotides or nucleotide bases refer to nucleoside phosphates. This includes natural, synthetic, or modified nucleotides, or a surrogate replacement moiety (e.g., inosine). The nucleoside phosphate may be a nucleoside monophosphate (NMP), a nucleoside diphosphate (NDP) or a nucleoside triphosphate (NTP). The sugar moiety in the nucleoside phosphate may be a pentose sugar, such as ribose. A nucleotide may be, but is not limited to, a deoxyribonucleoside triphosphate (dNTP) or a ribonucleoside triphosphate (rNTP).

Nucleotide analogues are compounds that are structurally similar to naturally occurring nucleotides. The nucleotide analogue may have an altered phosphate backbone, sugar moiety, nucleobase, or combinations thereof. It will be understood that the use of such analogues results in nucleic acids which may have different base-pairing properties and the interactions that occur when such bases are stacked may be different to those seen in natural nucleic acids.

The amplification reaction is preferably isothermal (at a constant temperature), unlike amplifications such as PCR which require temperature cycling. The methods may be used in the amplification of any appropriate template, preferably a circular nucleic acid template. The nucleic acid template can be provided in any appropriate amount to the reaction, including a minimal amount.

It is preferred that the nucleic acid template is amplified using RCA.

The polymerase enzyme or enzymes used for amplification may be a proofreading or a non-proofreading nucleic acid polymerase. The nucleic acid polymerase used may be a strand displacing nucleic acid polymerase. The nucleic acid polymerase may be a thermophilic or a mesophilic nucleic acid polymerase.

The method may require a highly processive, strand-displacing polymerase to amplify the nucleic acid template under conditions for high fidelity amplification. The fidelity of a polymerase is the result of accurate replication of the template. In addition to effective discrimination of correct versus incorrect nucleotide incorporation, some polymerases possess a 3′ to 5′ exonuclease activity. This proofreading activity is used to excise incorrectly incorporated bases that are then replaced with the correct one. High-fidelity amplification utilises polymerases that couple low misincorporation rates with proofreading activity to give faithful replication of the template.

The amplification reaction may employ a polymerase that generates single stranded, amplified nucleic acid after amplification. The polymerase is therefore capable of strand displacement synthesis.

A Phi29 DNA polymerase or Phi29-like polymerase may be used for amplifying a template in some embodiments. Alternatively, a combination of a Phi29 DNA polymerase and another polymerase may be used.

The amplification reaction may employ a low concentration of primer in one version of the method. The present inventors have found that a low concentration of primer is advantageous, since it enables the amplification reaction to generate only single stranded nucleic acid. A primer is a short linear oligonucleotide which hybridises to a sequence within the template to prime the nucleic acid synthesis reaction. The primer may be any nucleic acid, such as RNA, DNA, non-natural nucleic acid or a mixture of the same. The primer may contain natural, synthetic, or modified nucleotides.

Alternatively, assuming that the template is a double stranded circular template, a nicking enzyme may be employed to make a nick on one strand of the double stranded template. This leaves an entry point for the polymerase, which then utilises the nicked strand of the template itself to prime the nucleic acid synthesis reaction.

The nucleic acid template is therefore amplified by contacting the template with at least a polymerase and nucleotides and incubating the reaction mixture under conditions suitable for nucleic acid amplification. The amplification of the nucleic acid template may be performed under isothermal conditions. Additional components may include one or more of: a nicking enzyme (nickase), a cofactor (e.g. magnesium ions), a primer, and/or a buffering agent.

Rolling circle amplification of a circular template generates a linear single stranded concatemer with adjacent multiple repeats encoded by the template (each one called a sequence unit herein). Due to the nature of the template, this means that each sequence unit includes a sequence of interest flanked by a formatting element. This means that the sequence of interest has a formatting element at each end. Each sequence unit may also include backbone sequence.

This method relies upon a sequence encoding a formatting element within the template, one at each end of a sequence encoding the sequence of interest. This formatting element is two adjacent sequences encoding a processing motif and a conformational motif. A forward formatting element comprises a processing motif adjacent to a conformational motif, and a reverse formatting element comprises a conformational motif adjacent to a processing motif. The processing motif includes a recognition site for an endonuclease and an associated cleavage site.

The concatemer may be processed into the nucleic acid constructs using an endonuclease. The cleavage site releases the terminal residue of the conformational motif.

When the cleavage site in the concatemeric nucleic acid is cut by the requisite endonuclease, this releases the conformational motif from the processing motif, enabling the sequestering of the end of the single stranded nucleic acid molecule under the appropriate conditions.

The amplification and processing reactions may occur simultaneously, i.e. the endonuclease may be present to process the concatemer as soon as it is formed, or there may be a delay in adding the endonuclease until the amplification is further advanced, or indeed complete.

The method to make the single stranded nucleic acid constructs is therefore elegant and efficient, and not limited by length of the sequence of interest

Template

In the template, a sequence encoding the sequence of interest is flanked on both sides by a sequence encoding a formatting element. One is in the forward orientation, and the other is in the reverse orientation. The encoded sequence is nested, such that the sequence of interest is flanked by a conformational motif, which in turn is directly adjacent to a processing motif, the conformational motif and the processing motif together forming the formatting element. Such nesting can be represented as seen in FIG. 1. The sequences of the processing motif and the conformational motif are thus contiguous. Alternatively put, the formatting element at each end of the sequence of interest are in the opposite or mirrored orientation, ensuring that the conformational motif is closest to the sequence of interest, whilst the processing motif is the outermost part of the formatting element.

The formatting element is unique in the production of single stranded nucleic acid molecules, but is not present in complete form in the final product, since the processing motif is cleaved from the conformational motif. The action of the endonucleases during processing ensures that the cleavage site of the processing motif is cut, therefore discarding the processing motif. It is thus a mechanism by which to produce a useful product that is partially removed, ensuring that the final product contains the minimum amount of unnecessary sequences, providing more room for the sequence of interest. Thus, the processing motif and the adjacent conformational motif are effectively joined until the cleavage site is cut, releasing the terminal residue of the product. The combination of a processing motif adjacent to a conformational motif, effectively separated by a cleavage site for an endonuclease, enables the direct production of a single stranded nucleic acid with sequestered ends from a longer single stranded nucleic acid molecule in a single step process, using an endonuclease. The processing motif is removed from the single stranded nucleic acid via processing with a restriction enzyme, and is not present in the single stranded nucleic acid with sequestered ends.

The formatting element is effectively cleaved by the action of the endonuclease, and therefore partially removed from the final product.

Processing Motif

A processing motif includes sequences capable of forming a base-paired section including a recognition site for an endonuclease and an associated cleavage site. It will be appreciated that the cleavage site can be remote from the recognition site, but that both are generally required to be in a duplexed structure.

In one format, a processing motif may be capable of forming a base-paired section due to the inclusion of at least one region of sequence which is capable of binding to another sequence within the processing motif, these sections may be seen to be self-complementary in sequence. These sequences may be contiguous or may be separated by a spacer element. Such motifs may be designed by including complementary stretches of sequence in the single stranded nucleic acid. It will be appreciated that although both sequences are present on the same strand of nucleic acid, the design of the molecules ensures that one sequence is in the correct orientation to bind to the other, intramolecularly. For example, in DNA, the sequences need to run antiparallel in order for the base pairs to form. Such motifs are common amongst viral single stranded genomes, for example.

The base-paired section of a processing motif may be contiguous, such that the section forms a hairpin or the like. The nucleic acid may form antiparallel double stranded hairpin like structures. The hairpin structure consists of a double stranded base paired region called a stem. Alternatively the base-paired section of a processing motif may include a spacer sequence between the two stretches of sequence capable of base-pairing, such that structures such as stem-loops are formed. The spacer may be any suitable length. The hairpin may be formed of a nucleic acid sequence which is palindromic, as defined herein.

The base paired or double stranded section of the nucleic acid molecule can also have complementary sequence. Base pairing and duplexes are defined further herein.

In the base-paired section of a processing motif, there is included a recognition site for an endonuclease, and an associated cleavage site. It is preferred that the cleavage site forms at the footing of the base-paired section, such that the entire processing motif may be cleaved from the single strand using the requisite endonuclease.

The base-pairing occurs between at least two sections of sequence within the single strand. This base-pairing may be standard (i.e. Watson and Crick classical base pairs which are adenine (A)-thymine (T) in DNA, adenine (A)-uracil (U) in RNA, and cytosine (C)-guanine (G) in both) or non-canonical (i.e. Hoogsteen base pairs or interactions among carbon-hydrogen and oxygen/nitrogen groups and the like). These are described elsewhere.

The template includes one or more sequences encoding a processing motif with any of these characteristics. The processing motifs may be different sequences.

The template may contain a sequence encoding a first processing motif and a sequence encoding a second processing motif. Encoded by the template, the first and second processing motifs are positioned at the outside edge of the conformational motif (and within the formatting element), such that each end of the sequence of interest finishes with formatting elements that are in the opposite orientations (forward and reverse).

Given the nature of the requirements for the processing motif in the single stranded nucleic acid concatemer (prior to processing), the sequence of the first and second processing motifs may be the same or different. If they are the same, then the restriction site forms at the footing of the base-paired section, such that the entire processing motif may be cleaved from the single strand using the requisite endonuclease. Therefore, regardless of the orientation of the processing motif with respect to the sequence of interest (before or after) then the whole processing motif can be cleaved from the nucleic acid, since the cleavage site is at the footing of the base-paired section, which could also be described as the final base pair of the paired section, or the base thereof.

Alternatively, the first and second processing motifs in the single stranded nucleic acid concatemer (prior to processing) may be different, such that each recognition site for an endonuclease containing a cleavage site is also different, enabling the use of different endonucleases when processing the single stranded concatemer of the invention.

The template may therefore include sequences encoding identical or different first and second processing motifs.

An endonuclease is an enzyme, whether proteinaceous or composed of nucleic acid such as DNA, that cleave the phosphodiester bond within a polynucleotide chain. In this invention, a cut through double-stranded nucleic acid is required in order to produce the nucleic acid molecule with sequestered ends. Therefore, a combination of two endonucleases may be required, each one cutting through a single strand. Alternatively, a single enzyme that cleaves both strands may be employed. The endonuclease may be a nicking endonuclease, a homing endonuclease, a guided endonuclease such as Cas9, or a restriction endonuclease, for example. A nicking endonuclease may be a modified restriction endonuclease that has been modified to cut only one strand.

In one aspect, the endonuclease is a restriction endonuclease.

A restriction endonuclease is an enzyme that cleaves double stranded nucleic acid at cleavage sites within or near to a specific recognition site. To cut, all restriction endonucleases make two incisions, once through each backbone (i.e. each strand) of the duplex. Since a restriction endonuclease requires the presence of double stranded nucleic acid in order to recognise the recognition site, such a structure is required in order to allow the endonuclease to cleave the nucleic acid. Therefore, the present inventors propose the construction of a base-paired section within the single stranded nucleic acid, preferably using self-complementary sequences, such that the single stranded molecule forms a double stranded structure including the recognition and cleavage sites.

Restriction endonucleases recognize a specific sequence of nucleotides and produce a double-stranded cut in the duplex. The recognition site can also be classified by the number of bases, usually between 4 and 8 bases. Many, but not all, of the recognition sites are palindromic, and this property is very useful when designing the processing motif, since it aids the design of the sequence enabling it to be placed in a base-paired section more easily. In the single stranded format, each section that is capable of forming the palindrome when base-paired to each other is called inverted repeat sequences. These two sequences may be separated by a spacer sequence in the single stranded nucleic acid.

The restriction endonuclease may be a blunt cutter (i.e. cut straight through the base-paired section) or cut in an offset fashion (i.e. cut is staggered through the base-paired section). The cleavage site can be within the recognition site, or nearby, and thus the cleavage site does not need to be part of the recognition site. Therefore, the cleavage site is associated with the recognition site, but does not necessarily form part of it.

Many thousands of restriction endonucleases are known, both natural and engineered, together with their recognition and cleavage sites. Any suitable recognition and cleavage sites may be included in a processing motif. Exemplary restriction endonucleases commonly used in cloning and the like are HhaI, HindIII, NotI, EcoRI, ClaI, BamHI, BglII, DraI, EcoRV, Pst1, SalI, SmaI, SchI and XmaI. Many are commercially available from suppliers such as New England Biolabs and ThermoFisher Scientific.

In order for the cleavage using the endonuclease to release the conformational motif from the formatting element in the single stranded nucleic acid concatemer, it is preferred that the cleavage site is adjacent to the conformational motif in the template, such that the terminal nucleotide of the conformational motif forms the terminal and sequestered end of the single stranded nucleic acid molecule product.

Within the template, encoded is a formatting element, one part of which is a sequence encoding a conformational motif, which is designed to be folded in the final single stranded nucleic acid molecule with sequestered ends. The conformational motif sequesters the ends (i.e. 5′ and 3′ ends for DNA and RNA) of the single stranded nucleic acid molecule.

A conformational motif includes sequences capable of forming a base paired section or duplex within the single stranded nucleic acid molecule or with a capping oligonucleotide. This base-paired section or duplex may form in the concatemer prior to processing with an endonuclease, or it may form after processing with an endonuclease, once the processing motif has been removed from the concatemer. Referring to the Figures, these have been depicted with the conformational motif forming a base-paired section in the concatemeric nucleic acid (see FIGS. 2, 3, 4 and 5) for ease of reference. These structures may not form until the processing motif has been cleaved by the endonuclease. If a capping oligonucleotide is required, the conformational motif will fold appropriately when this is added, since base-pairing to this entity can cause the requisite folding.

The duplex may be formed by base-pairing between at least two sections of sequence within the single strand. This base-pairing may be standard (i.e. Watson and Crick classical base pairs which are adenine (A)-thymine (T) in DNA, adenine (A)-uracil (U) in RNA, and cytosine (C)-guanine (G) in both) or non-canonical (i.e. Hoogsteen base pairs, interactions among carbon-hydrogen and oxygen/nitrogen groups and the like). Hoogsteen pairs allow formation of particular structures of single stranded nucleic acid G-rich segments called G-quadruplexes, or C-rich segments called i-motifs. G quadruplexes generally require four triplets of G, separated by short spacers. This permits assembly of planar quartets which are composed of stacked associations of Hoogsteen bonded guanine molecules.

A conformational motif may therefore include sections of sequence which are self-complementary or complementary to another sequence within single stranded nucleic acid molecule, i.e. to the sequence of interest or a spacer sequence within the sequence of interest.

A conformational motif may include sequences for forming more than one base-paired section or duplex, each of which are separated by spacer sequences of single stranded nucleic acid, or the base paired sections or duplexes may form part of larger structures which may include any one or more of the following: hairpin; single stranded regions; bulge loop; internal loop; multi-branched loop or junction.

Once the conformational motif has formed at least one base-paired section or duplex, the terminal residue of the single stranded nucleic acid molecule is sequestered. The terminal nucleotide (or residue) at either end of the single stranded DNA is tucked away/protected. This renders the terminal residues to be not readily available to single strand exonuclease and the like.

The terminal nucleotide of the single stranded nucleic acid molecule is sequestered, either by being included within the base-paired section or duplex of the conformational motif, and thus lacking a free single stranded terminal end, or folded within the topology of the conformational motif, such that the terminal end is not free for further interaction, and is secured.

It is preferred that the terminal end (terminal nucleotide) is not in single stranded form in the single stranded nucleic acid product. These ends are stabilised by presence of base pairing between each terminal residue and another part of the single stranded nucleic acid.

A conformational motif from the concatemeric nucleic acid molecule, once processed, forms one end of the single stranded nucleic acid construct. The terminal residue is sequestered by the conformational motif.

In the single stranded nucleic acid construct, each end is sequestered by a conformational motif.

Preferred conformational motifs according to the present invention include sequences which can fold as hairpins, stem loops, junctions, pseudoknots, ITRs, modified ITRs, synthetic ITRs, i-motifs and G-quadruplexes.

A hairpin is a structure in a nucleic acid, such as DNA or RNA, due to base-pairing between neighbouring complementary sequences of a single strand of the nucleic acid. The neighbouring complementary sequences may be separated by a few nucleotides, e.g. 1-10 or 1-5 nucleotides. An example of this is depicted in FIG. 2. If a loop of non-complementary sequence is included between the two sections of complementary sequence, this forms a hairpin loop or a stem loop. The loop may be of any suitable length, as may the stem or double stranded section. Other similar structures include lariats.

The conformational motifs at each end can fold into the same particular structure (i.e. a hairpin, stem loop, ITR or the like) or they can each independently be designed to fold into different structures (i.e. the first end is a hairpin and the second end is a ITR).

As discussed previously, the conformational motifs can have additional function. They can form functional structures such as aptamers and the like. Alternatively, they can be designed provide a mechanism to bind the single stranded nucleic acid constructs together in oligomeric conformations.

The template also encodes for a sequence of interest. In the concatemer and single stranded nucleic acid construct with sequestered ends, the sequence of interest can be any desired nucleic acid sequence, of any suitable length. The sequence of interest may be a functional sequence (i.e. directly act as an aptamer or the like without further transcription or translation). Alternatively, the sequence of interest can encode a functional sequence. Functional sequences include aptamers, catalytic entities due as nucleic acid enzymes including ribozymes, non-coding RNA (ncRNA) including microRNAs (miRNAs), short interfering RNAs (siRNAs), and piwi-interacting RNAs (piRNAs).

The sequence of interest may be capable of acting as donor nucleic acid for gene editing purposes, both in animals and plants. Exemplary methods of gene editing include CRISPR gene editing and Transcription activator-like effector nucleases (TALENs) based methods. If the sequence of interest is to be a donor nucleic acid, it may be necessary to include sequences or elements to enable the excision of the donor nucleic acid by the necessary machinery.

The sequence of interest may be a transgene, such as a gene or genetic material, for expression in a cell. The transgene is operably connected to a promoter sequence within an expression cassette.

The sequence of interest may include a sequence which encodes a therapeutic product. The therapeutic product may be a DNA aptamer, a protein, a peptide, or an RNA molecule, such as small interfering RNA. In order to provide for therapeutic utility, such a sequence of interest may comprise an expression cassette comprising one or more promoter or enhancer elements and a gene or other coding sequence which encodes an mRNA or protein of interest. The expression cassette may comprise a eukaryotic promoter operably linked to a sequence encoding a protein of interest, and optionally an enhancer and/or a eukaryotic transcription termination sequence.

The sequence of interest may be used for production of DNA for expression in a host cell, particularly for production of DNA vaccines. DNA vaccines typically encode a modified form of an infectious organism's DNA. DNA vaccines are administered to a subject where they then express the selected protein of the infectious organism, initiating an immune response against that protein which is typically protective. DNA vaccines may also encode a tumour antigen in a cancer immunotherapy approach. Any DNA vaccine may be used as the sequence of interest.

Also, the process of the invention may produce other types of therapeutic DNA molecules e.g. those used in gene therapy. For example, such DNA molecules can be used to express a functional gene where a subject has a genetic disorder caused by a dysfunctional version of that gene. Examples of such diseases are well known in the art.

It is preferred that the portion of the template encoding the sequence of interest or the conformational motif lacks a bacterial origin of replication, lacks resistance genes (i.e. for antibiotics), lacks CpG islands (except for DNA vaccines where the same may be helpful), lacks methylation of cytosine and adenine or any other marker of foreign DNA. These entities can, however, be present outside the sequence of interest and conformational motif, since the rest of the template is processed and removed from the product.

The template is preferably circular or capable of circularisation. The template may be double stranded or single stranded.

If the template is double stranded, it is preferred that it includes a sequence for a nicking enzyme prior to the first processing motif. Alternatively known as nicking endonucleases, these enzymes hydrolyse only one strand of the duplex, to produce nucleic acid molecules that are “nicked”, rather than cleaved. This provides a start-point for rolling circle amplification without the need for additional primer and can ensure that only one strand of nucleic acid concatemer is produced in the amplification reaction. Such enzymes are commercially available, for example from New England Biolabs and Thermo Fisher Scientific. These enzymes are specific enough such that a recognition and cleavage site can be designed on the relevant strand of the template to ensure the correct strand is used directly as the template.

The template may be any suitable nucleic acid, either natural such as DNA or RNA, or artificial as discussed previously. It is preferred that the template is DNA.

Amplification of the Template

In order to produce the single stranded nucleic acid constructs, the template has to be amplified enzymatically.

The template may be amplified with one or more polymerase enzymes. The polymerase enzyme can use the template to synthesise a complementary nucleic acid copy, if provided with sufficient raw materials or substrates (such as nucleotides) and co-factors (such as metal ions and the like) in order to amplify the nucleic acid.

Any suitable polymerase enzyme may be used for this amplification step, and it is possible to use one enzyme, or a combination of enzymes.

The enzyme may be a DNA polymerase or RNA polymerase depending on the nature of the template, or an artificial, modified, engineered or mutant polymerase in order to use a synthetic template or to manufacture a synthetic single stranded nucleic acid.

Amplification is preferred to proceed via strand displacement methods. This is an isothermal method that does not require repeated cycles of heating and cooling (as PCR does), but the polymerase enzyme is capable of displacing any strand which is annealed to the template. Strand-displacement type polymerases are known, including Phi29, Deep Vent®, BST DNA polymerase I and variants of the same. This means that multiple polymerases can act on the same template at the same time, each one displacing the nascent strand produced by the earlier polymerase.

The most preferred strand displacement amplification technique is rolling circle amplification (RCA). In this method of amplification, strand displacing polymerases progress continually around a circular template whilst extending the nascent oligonucleotide. This leads to the generation of long concatemeric strands of nucleic acid.

It is preferred that the amplification reaction is allowed to initiate on a double stranded circular template by nicking the template with a nicking endonuclease. Such enzymes are discussed above. By nicking a single strand of a double stranded template, this opens up the template for the polymerase to bind, and it may utilise the free 3′ end created to extend this strand into a concatemeric nucleic acid by processing around the circular template many times.

The use of a nicking site in the template and a nicking endonuclease also permits the method only to make a single stranded concatemer from the RCA, and prevents the amplification of the opposite strand, since only one backbone is cleaved using the enzyme.

Thus, the use of a nicking site in the template is preferred, since it allows for the production of the desired product, and prevents the unwanted amplification of the complementary strand of a double stranded template.

Alternatively, the present inventors have found that using a very low quantity of a specific primer which is designed to anneal to the desired template strand (and not its complementary strand), that the amplification can be forced to proceed to make large quantities of only one strand of a double stranded template. In this aspect, only picoMolar quantities of primer are required. Thus, the primer may be supplied in a quantity of 1 pM to 100 nM.

If the template is single stranded, then it is possible to use a primer to initiate the rolling circle amplification. Preferably, the primer is designed only to anneal to the template and not to the concatemeric nucleic acid molecule, thus ensuring that only one species of concatemer is made.

The inventors have therefore devised ways of ensuring that RCA proceeds to amplify a template and produce only the desired concatemer, the correct species for the production of single stranded nucleic acid constructs, and not the complementary strand. Making the complementary strand would result in a 50% waste amplification reaction and also make the synthesis of single stranded constructs much more difficult, since the presence of complementary concatemers would inherently result in the formation of double stranded nucleic acid.

The template is contacted with at least one polymerase. One, two, three, four or five different polymerases may be used. The polymerase may be any suitable polymerase, such that it synthesises polymers of nucleic acid. The polymerase may be a DNA or RNA polymerase. Any polymerase may be used, including any commercially available polymerase. Two, three, four, five or more different polymerases may be used, for example one which provides a proofreading function and one or more others which do not. Polymerases having different mechanisms may be used e.g. strand displacement type polymerases and polymerases replicating nucleic acid by other methods. A suitable example of a DNA polymerase that does not have strand displacement activity is T4 DNA polymerase.

A polymerase may be highly stable, such that its activity is not substantially reduced by prolonged incubation under process conditions. Therefore, the enzyme preferably has a long half-life under a range of process conditions including but not limited to temperature and pH. It is also preferred that a polymerase has one or more characteristics suitable for a manufacturing process. The polymerase preferably has high fidelity, for example through having proofreading activity. Furthermore, it is preferred that a polymerase displays high processivity, high strand-displacement activity and a low Km for nucleotides and nucleic acid. A polymerase may be capable of using circular and/or linear DNA as template. The polymerase may be capable of using double stranded or single stranded nucleic acid as a template. It is preferred that a polymerase does not display exonuclease activity that is not related to its proofreading activity.

The skilled person can determine whether or not a given polymerase displays characteristics as defined above by comparison with the properties displayed by commercially available polymerases, e.g. Phi29 (New England Biolabs, Inc., Ipswich, Mass., US), Deep Vent® (New England Biolabs, Inc.), Bacillus stearothermophilus (Bst) DNA polymerase I (New England Biolabs, Inc.), Klenow fragment of DNA polymerase I (New England Biolabs, Inc.), M-MuLV reverse transcriptase (New England Biolabs, Inc.), VentR® (exo-minus) DNA polymerase (New England Biolabs, Inc.), VentR® DNA polymerase (New England Biolabs, Inc.), Deep Vent® (exo-) DNA polymerase (New England Biolabs, Inc.), Bst DNA polymerase large fragment (New England Biolabs, Inc.), hi-fidelity fusion DNA polymerase (e.g., Pyrococcus-Yke, New England Biolabs, MA), Pfu DNA polymerase from Pyrococcus furiosus (Strategene, Lajolla, Calif.), Sequenase™ variant of T7 DNA polymerase, T7 DNA polymerase, T4 DNA polymerase, DNA polymerase from Pyrococcus species GB-D (New England Biolabs, MA), or DNA polymerase from Thermococcus litoralis (New England Biolabs, MA).

Alternatively, the polymerase may be a DNA-dependent RNA polymerase. Exemplary enzymes include T3 RNA Polymerase, T7 RNA Polymerase, Hi-T7™ RNA Polymerase, SP6 RNA Polymerase, E. coli Poly(A) Polymerase, E. coli RNA Polymerase, and E. coli RNA Polymerase, Holoenzyme (all available from NEB).

Where a high processivity is referred to, this typically denotes the average number of nucleotides added by a polymerase enzyme per association/dissociation with the template, i.e. the length of primer extension obtained from a single association event.

Strand displacement-type polymerases are preferred. Preferred strand displacement-type polymerases are Phi 29, Deep Vent and Bst DNA polymerase I or variants of any thereof. “Strand displacement” describes the ability of a polymerase to displace complementary strands on encountering a region of double stranded DNA during synthesis. The template is thus amplified by displacing complementary strands and synthesizing a new complementary strand. Thus, during strand displacement replication, a newly replicated strand will be displaced to make way for the polymerase to replicate a further complementary strand. The amplification reaction initiates when a primer or the free end of a single stranded template anneals to a complementary sequence on a template (both are priming events). When nucleic acid synthesis proceeds and if it encounters a further primer or other strand annealed to the template, the polymerase displaces this and continues its strand elongation. It should be understood that strand displacement amplification methods differ from PCR-based methods in that cycles of denaturation are not essential for efficient amplification, as double-stranded template is not an obstacle to continued synthesis of new strands. Strand displacement amplification may only require one initial round of heating, to denature the initial template if it is double stranded, to allow the primer to anneal to the primer binding site if used. Following this, the amplification may be described as isothermal, since no further heating or cooling is required. In contrast, PCR methods require cycles of denaturation (i.e. elevating temperature to 94 degrees centigrade or above) during the amplification process to melt double-stranded DNA and provide new single stranded templates. During strand displacement, the polymerase will displace strands of already synthesised nucleic acid.

A strand displacement polymerase used in the process of the invention preferably has a processivity of at least 20 kb, more preferably, at least 30 kb, at least 50 kb, or at least 70 kb or greater. In one embodiment, the strand displacement DNA polymerase has a processivity that is comparable to, or greater than phi29 DNA polymerase.

The contacting of the template with the polymerase and either a nickase or a primer may take place under conditions promoting annealing of primers to the template. The conditions include the presence of single-stranded DNA allowing for hybridisation of the primers. The conditions also include a temperature and buffer allowing for annealing of the primer to the template. Appropriate annealing/hybridisation conditions may be selected depending on the nature of the primer. An example of preferred annealing conditions used in the present invention include a buffer 30 mM Tris-HCl pH 7.5, 20 mM KCl, 8 mM MgCl₂. The annealing may be carried out following denaturation using heat by gradual cooling to the desired reaction temperature.

The template and polymerase are also contacted with nucleotides. The combination of template, polymerase and nucleotides forms a reaction mixture. The reaction mixture may also comprise a one or more primers or alternatively a nicking enzyme (nickase). The reaction mixture may independently also include one or more metal cations or any other required co-factors for nucleic acid synthesis.

A nucleotide is a monomer, or single unit, of nucleic acids, and nucleotides are composed of a nitrogenous base, a five-carbon sugar (ribose or deoxyribose), and at least one phosphate group. Any suitable nucleotide may be used.

The nucleotides may be present as free acids, their salts or chelates, or a mixture of free acids and/or salts or chelates.

The nucleotides may be present as monovalent metal ion nucleotide salts or divalent metal ion nucleotide salts.

The nitrogenous base may be adenine (A), guanine (G), thymine (T), cytosine (C), and/or uracil (U). The nitrogenous base may also be modified bases, such as 5-methylcytosine (m5C), pseudouridine (Ψ), dihydrouridine (D), inosine (I), and/or 7-methylguanosine (m7G).

It is preferred that the five-carbon sugar is a deoxyribose, such that the nucleotide is a deoxynucleotide.

The nucleotide may be in the form of deoxynucleoside triphosphate, denoted dNTP. This is a preferred embodiment of the present invention. Suitable dNTPs may include dATP (deoxyadenosine triphosphate), dGTP (deoxyguanosine triphosphate), dTTP (deoxythymidine triphosphate), dUTP (deoxyuridine triphosphate), dCTP (deoxycytidine triphosphate), dITP (deoxyinosine triphosphate), dXTP (deoxyxanthosine triphosphate), and derivatives and modified versions thereof. It is preferred that the dNTPs comprise one or more of dATP, dGTP, dTTP or dCTP, or modified versions or derivatives thereof. It is preferred to use a mixture of dATP, dGTP, dTTP and dCTP or modified version thereof.

The nucleotides may be in solution or provided in lyophilised form. A solution of nucleotides is preferred.

The nucleotides may be provided in a mixture of one or more suitable bases, including any newly designed artificial bases, preferably, one or more of adenine (A), guanine (G), thymine (T), cytosine (C). Two, three or preferably all four nucleotides (A, G, T, and C) are used in the process to synthesise the nucleic acid.

Concatemer

The single stranded concatemer produced is also new, and is capable of being processed into single stranded nucleic acid with sequestered ends, which can contain a sequence of interest.

The concatemer is a nucleic acid molecule with repeated units of the sequence unit present in the template. Each sequence unit includes a sequence of interest flanked on both sides by formatting elements, as described previously. The sequence unit may also include backbone sequence encoded by the template, which is ultimately not present in the nucleic acid construct of the invention.

Concatemeric nucleic acid molecules may comprise multiple sequence units, for example, 10, 50, 100, 200, 500 or even 1000 or more sequence units in continuous series. Concatemeric molecules may be at least 5 kb in size, at least 50 kb, at least 100 kB, or even up to 200 kB in length.

Processing the Concatemeric Nucleic Acid Molecule

Once the template has been amplified, or even during amplification, the concatemeric nucleic acid may be processed into single stranded nucleic acid constructs using the requisite endonucleases which will cleave the one or more processing sites.

It is therefore preferred that the processing motif is capable of forming a base-paired portion whilst in the form of a concatemeric nucleic acid. Thus, the processing motif may be designed such that the base pairs form under the conditions suitable for isothermal amplification. Once these base-paired portions have formed within the concatemeric nucleic acid, recognition sites for the endonucleases form, together with the necessary cleavage sites. This elegant system allows for the processing of the concatemer, despite the fact that it is only a single strand of nucleic acid. It is the design of the template that allows for the formation of processing sites within the concatemeric nucleic acid, allowing for a single step to process this concatemer by the addition of one or more endonucleases.

The endonucleases may be added once the amplification reaction is complete, whilst it is underway or at the start of the amplification reaction. It is preferred that the amplification reaction is underway before the endonucleases are added, to ensure that the concatemeric nucleic acid is processed quickly. Alternatively, the amplification process may be allowed to complete (i.e. Template exhausted, nucleotides exhausted, reaction mixture too viscous) prior to the addition of endonucleases.

Once cleaved with the endonucleases, the concatemer is cut into single stranded nucleic acid constructs with sequestered ends thanks to the action of the conformational motifs. Also produced are side products that consist of the processing motif plus any associated template backbone. Since the ends of the side products are not sequestered, these may be removed using a single stranded exonuclease.

The invention will now be described with reference to the following non-limiting examples.

### EXAMPLES

### Example 1: Production of Nucleic Acid Construct

Template: Template A (FIG. 9, SEQ ID No. 1).

The template includes a nicking site, a processing motif adjacent to a conformational motif, a sequence of interest, a second conformational motif adjacent to a second processing motif, and a backbone of similar size to the sequence of interest. There is an additional endonuclease target site in the backbone, which will only cut in dsDNA.

Sequence of template A is presented as SEQ ID No. 1 in the associated sequence listing.

Nicking Reaction in 20 μl
4 μl template (stock concentration of 1 μg/μl)
13 μl water
2 μl CutSmart buffer (NEB)
1 μl nickase (Nb.BsrDI, NEB)
Incubated for 180 minutes at 37° C., followed by 20 minutes at 80° C.

Amplification Reaction in 1000 μl
4 μl template (stock concentration 0.2 μg/μl)
100 μl buffer—10× stock solution:
   300 mM Tris pH 7.9
   300 mM KCl
   50 mM (NH₄)₂SO₄
   100 mM MgCl₂
837 μl ddH₂O
20 μl dNTPs (stock solution 100 mM (Bioline))
35 μl SSB (stock solution 5 μg/μl (E. coli SSB, in-house preparation))
2 μl inorganic pyrophosphatase (stock solution 2 U/μl (Enzymatics))
2 μl phi29 DNA polymerase (stock solution 100 U/μl (Enzymatics))
Incubated for 16 hours at 30° C.

Processing Reaction
1000 μl amplification reaction
20 μl MlyI (stock solution 10 U/μl)
Incubated for 180 minutes at 37° C.

Result:

Gel photograph shown in FIG. 10.

This gel shows the digested product of the RCA reaction. Left hand well: Thermo Scientific Gene Ruler 1 kb Plus DNA ladder (sizes in bp on the left). Right-hand well: MlyI processed RCA (expected sizes in nt (nucleotides) on the right). The backbone and product bands, which are of similar size, do not stain brightly due to their primarily single-stranded nature. No ‘signature’ lower band is seen which would indicate double-stranding of the product (an MlyI site exists in the backbone, and would cut in dsDNA to drop the backbone band down to 1597 and 407 base pairs).

### Example 2: Testing the Stability of the Terminal Nucleotides of Nucleic Acid Constructs with Exonuclease

This Example tests if the novel nucleic acid constructs with sequestered ends offer significant exonuclease resistance in comparison with nucleic acid whose ends do not form a defined structure (standard single-stranded DNA).

Exonuclease Stability Test:

Five product molecules were generated for this test, with different conformational motifs:
i. ssDNA with no conformational motifs (and therefore unsecured terminal nucleotides)
ii. ssDNA with a trinucleotide loop (GAA) conformational motif securing the terminal nucleotide at both the 3′ and 5′ ends in a stretch of base-paired duplex;
iii. ssDNA with a G-quadruplex conformational motif (TTAGGG)₄ (SEQ ID No. 11) together with an additional sequence which forms a section of intramolecular base-pairing to the sequence of interest and includes the terminal nucleotide within a section of duplex nucleic acid;
iv. ssDNA with a G-quadruplex conformational motif without an additional base-pairing section at both the 3′ and 5′ ends, thus relying on securing each terminal nucleotide by embracing it within the quadruplex (TTAGGG)₄;
v. ssDNA with a pseudo-knot conformational motif without an additional base-pairing section at both the 3′ and 5′ ends, thus relying on securing each terminal nucleotide by its incorporation within the pseudoknot.

The nucleic acid molecules were diluted to 100 ng/μl in 100 mM KCl, and were heat denatured (95° C., and cooled to room temperature) to allow the conformational motifs to form conformations as appropriate. 10 μl of each of construct was used for subsequent exonuclease tests in 50 μl final volume in 1× exonuclease VII reaction buffer (NEB; 50 mM Tris-HCl, 50 mM Sodium Phosphate, 8 mM EDTA, 10 mM 2-mercaptoethanol, pH 8.0). Reactions were incubated at 37° C. for 30 minutes in the presence or absence of 100 U/ml of Exonuclease VII (NEB). Products were resolved on an agarose gel with GelRed dye (FIG. 6).

TABLE 1
Reagents

| | | Stock | | | | Final reaction | |
|---|---|---|---|---|---|---|---|
| | Reagent | concentration | | Volume | | concentration | |
| Denaturation | DNA | 2500 | ng/μl | 4 | μl | 100 | ng/μl |
| mix | | | | | | | |
| | KCl | 1M | | 10 | μl | 100 | mM |
| | H₂O | | | 86 | μl | to 100 μl total | |
| Reaction | DNA | 100 | ng/μl | 10 | μl | 20 | ng/μl |
| mix | | | | | | | |
| | Exonuclease | 5 x | | 10 | μl | 1 x | |
| | buffer | | | | | | |
| | H₂O | | | 29.5 | μl | to 50 μl total | |
| Enzyme | Exonuclease | 10 | U/μl | 0.5 | μl | 0.1 | U/μl |
| | VII | | | | | | |

TABLE 2
Materials:

| | | | |
|---|---|---|---|
| 1 kb ladder | NEB | N0468S | 0471511 |
| 6x gel loading dye | NEB | B7024S | 0361604 |
| Agarose LE | Cleaver Scientific | CSL-AG500 | 14150916 |
| Gel extraction kit | Promega | A9282 | 0000232671 |
| GelRed | Biotum | 41003 | 16G1010 |
| TAE buffer | IH | NA | NA |

Results:

ssDNA without conformational motifs securing the 3′ and 5′ ends was almost entirely digested in the presence of exonuclease VII within the short window of the experiment (FIG. 6, lanes 1-2).

All ssDNA which included a conformational motif to secure the 3′ and 5′ terminal nucleotides (as described in (ii) to (v) above), i.e. single stranded nucleic acid constructs with sequestered ends, were more resistant to exonuclease digestion than ssDNA.

The construct described as (ii) (lanes 3-4) sequestered the end by including it within a base-paired duplex stretch of sequence. This showed resistance to exonuclease.

Two different nucleic acid constructs were made using G-quadruplex conformational motifs. The construct described in (iv) (lanes 7-8) sequestered the end by embracing it within a G-quadruplex. The construct described in (iii) (lanes 5-6) includes an additional section of duplexed nucleic acid in which the terminal nucleotide is involved in base-pairing. For this experiment, it appeared that the addition of an extra duplex sequence assisted in the resistance to exonuclease. This demonstrates that the conformation can be engineered to suit the particular conditions under which the nucleic acid construct may be used, based upon the desired characteristics of the sequestered ends.

The construct described as (v) (lanes 9-10) sequestered the end by including it within a pseudoknot. This appeared to be display moderate resistance to exonuclease under the tested conditions.

These data show that sequestering the ends can be used to delay degradation by exonucleases and by changing the sequence of the conformational motif, the structure of the construct can be engineered to increase stability of the nucleic acid construct.

### Example 3: Testing the Stability of the Terminal Nucleotides of Nucleic Acid Constructs in the Presence of Cell Extract

This experiment was designed to test if novel nucleic acid constructs with sequestered ends offer significant resistance in the presence of cell extract in comparison with nucleic acid whose ends do not form a defined conformation (standard single-stranded DNA in these examples).

Cell Extract Preparation:

HEK293T cells (Clontech Z2180N) were grown in Eagle's minimal essential medium (supplemented with 10% FBS, glutamine, non-essential amino acids, and antibiotics) at 37° C. and 5% CO₂. Three 10 cm plates with full confluency were washed with PBS. Cells were harvested and lysed using 10 ml of 1× cell lysis buffer (Promega E397A). Approximately 2,000,000 cells per ml of suspension were obtained. After a 5 minute incubation at room temperature, the suspension was cleared by centrifugation (4000 rpm for 5 minutes). Glycerol was added to 20% and cell extract was aliquoted and frozen at −80° C.

Cell Extract Stability Test:

All 5 nucleic acid constructs (as prepared in Example 2) were diluted to 100 ng/μl in 100 mM KCl, and were heat denatured (95° C., and cooled to room temperature) to allow the conformational motifs to form conformations as appropriate. The dilutions were supplemented with 2 mM MgCl₂ and 10 mM Tris pH 7.5, and 5% of thawed cell extract. Samples were incubated for 24 or 72 hours, and products were resolved on an agarose gel with GelRed dye (FIG. 7).

TABLE 3
Reagents

| | | Stock | | | | Final reaction | |
|---|---|---|---|---|---|---|---|
| | Reagent | concentration | | Volume | | concentration | |
| Denaturation | DNA | 2500 | ng/μl | 4 | μl | 100 | ng/μl |
| mix | | | | | | | |
| | KCl | 1M | | 10 | | 100 | mM |
| | H₂O | | | 86 | μl | to 100 μl total | |
| Reaction | DNA | 100 | ng/μl | 20 | μl | 20 | ng/μl |
| mix | | | | | | | |
| | Cell extract | 100% | | 5 | μl | 5% | |
| | KCl | 1M | | 8 | μl | 100 | mM |
| | Tris 7.5 | 1M | | 1 | μl | 10 | mM |
| | MgCl₂ | 200 | mM | 1 | μl | 2 | mM |

| | | | |
|---|---|---|---|
| Water | 65 | μl | to 100 μl total |

TABLE 4
Materials

| | | | |
|---|---|---|---|
| 1 kb ladder | NEB | N0468S | 0471511 |
| 6x gel loading dye | NEB | B7024S | 0361604 |
| Agarose LE | Cleaver | CSL-AG500 | 14150916 |
| | Scientific | | |
| Gel extraction kit | Promega | A9282 | 0000232671 |
| GelRed | Biotum | 41003 | 16G1010 |
| TAE buffer | IH | NA | NA |
| L-Glutamine | Gibco | 25030-081 | 1817540 |
| MEM non-essential | Sigma | M7145-100ml | RNBG2199 |
| amino acid solution | | | |
| Minimum essential | Sigma | M2279-500ml | RNBG4545 |
| medium Eagle | | | |
| PBS | Sigma | D1408-100ML | RNBF3311 |
| Glycerol | Fisher | BP229-1 | 144356 |
| Reporter lysis | Promega | E397A | 0000264994 |
| buffer 5x | | | |

Results:

ssDNA lacking conformational motifs to sequester the 3′ and 5′ ends was gradually digested to near completion (lanes 1, 6 and 11) in the presence of 5% cell extract, and low amounts were detectable after 72 h of incubation.

All other nucleic acid constructs with sequestered ends offered significantly greater stability in the presence of the extract.

Under the conditions tested, it appears that sequestering the 3′ and 5′ terminal ends by inclusion within a section or stretch of duplex nucleic acid formed by base-pairing offered the greatest amount of resistance to degradation. The results for constructs (ii) and (iii) in lanes 2, 7, 12, and lanes 3, 8, 13, respectively, showed the greatest stability.

However, the remaining constructs showed some degree of resistance, demonstrating that it is possible to secure the terminal residue without it being directly involved in a base-pair. The version of G-quadruplex denoted (iv) displayed relatively strong stability (lanes 4, 9, 14), whilst the level of resistance to degradation of the molecule whose conformational motifs assumed pseudoknot structures (v) (lanes 5, 10, 15), was the lowest of the sequestered-ended constructs.

To eliminate the possibility that certain bands appeared as artefacts from cell extract, a control containing 5% extract without DNA added was incubated for 72 h (lane 16).

1. A nucleic acid template for the cell-free, in vitro manufacture of single stranded nucleic acid constructs with sequestered ends, comprising a sequence encoding the following elements from 5′ to 3′:
i) a first processing motif, adjacent to
ii) a first conformational motif,
iii) a sequence of interest,
iv) a second conformational motif, adjacent to
v) a second processing motif,
wherein a processing motif includes sequences capable of forming a base-paired section including a recognition site for an endonuclease and an associated cleavage site; and
wherein the conformational motif includes at least one sequence capable of forming intramolecular hydrogen bonds.

2. A nucleic acid template as claimed in claim 1 wherein the cleavage site within a processing motif is adjacent to a conformational motif.

3. A nucleic acid template as claimed in claim 1 wherein the cleavage site for the endonuclease within a processing motif is at the terminal base pair of the base-paired section.

4. A nucleic acid template as claimed in claim 1 wherein a conformational motif includes a sequence capable of forming intramolecular hydrogen bonds which act to secure the terminal nucleotide at the end of the single stranded nucleic acid construct.

5. A nucleic acid template as claimed in claim 1 wherein the single stranded nucleic acid construct includes any one or more of the following:
i) an aptamer;
ii) a nucleic acid enzyme;
iii) a guide sequence for gene editing;

6. A nucleic acid template as claimed in claim 1 wherein said intramolecular hydrogen bonds form between the nucleotide bases in the sequence of the conformational motif, and optionally allow the conformational motif to assume a conformation.

7. A nucleic acid template as claimed in claim 6 wherein the intramolecular hydrogen bonds between the nucleotide bases involve Watson-Crick base pairs, Hoogsteen base-pairs or noncanonical base-pairing.

8. A nucleic acid template as claimed in claim 1 wherein a conformational motif may be a sequence which is capable of assuming one or a combination of two or more of the following conformations:
i) quadruplex;
ii) hairpin;
iii) cruciform;
iv) stem loop; and/or
v) pseudoknot.

9. A nucleic acid template as claimed in claim 1 wherein the sequestered end involves the intramolecular base-pairing of the terminal nucleotide.

10. A nucleic acid template as claimed in claim 1 wherein the sequestered end involves including the terminal nucleotide in an ITR structure with a double stranded D region.

11. A method of manufacturing single stranded nucleic acid molecules with sequestered ends, comprising:
(a) amplifying a circular template using a polymerase capable of rolling circle amplification,
wherein said template comprises a sequence encoding the following elements:
i) a first processing motif, adjacent to
ii) a first conformational motif,
iii) a sequence of interest,
iv) a second conformational motif, adjacent to
v) a second processing motif,
wherein a processing motif includes sequences capable of forming a base-paired section including a recognition site for an endonuclease containing a cleavage site, and
wherein the conformational motif includes at least one sequence capable of forming intramolecular hydrogen bonds, said amplification producing a nucleic acid concatemer, and
(b) processing said nucleic acid concatemer using one or more endonucleases which recognise the cleavage sites in one or more of said processing motifs.

12. The method of manufacturing single stranded nucleic acid molecules with sequestered ends as claimed in claim 11, wherein the template is as described in claim 1.

13. A single stranded nucleic acid concatemer with two or more repeats of a sequence unit, said sequence unit comprising the following elements:
i) a first processing motif, adjacent to
ii) a first conformational motif,
iii) a sequence of interest,
iv) a second conformational motif, adjacent to
v) a second processing motif,
wherein a processing motif includes sequences capable of forming a base-paired section including a recognition site for an endonuclease and an associated a cleavage site, and wherein the conformational motif includes at least one sequence capable of forming intramolecular hydrogen bonds.

14. A single stranded linear nucleic acid molecule with sequestered ends wherein at least one sequestered end forms a G quadruplex.

15. A single stranded linear nucleic acid molecule with sequestered ends wherein at least one sequestered end forms an ITR structure with a double stranded D section.

16. A nucleic acid template as claimed in claim 2 wherein a conformational motif includes a sequence capable of forming intramolecular hydrogen bonds which act to secure the terminal nucleotide at the end of the single stranded nucleic acid construct.

17. A nucleic acid template as claimed in claim 3 wherein a conformational motif includes a sequence capable of forming intramolecular hydrogen bonds which act to secure the terminal nucleotide at the end of the single stranded nucleic acid construct.

18. A nucleic acid template as claimed in claim 4 wherein the sequestered end involves the intramolecular base-pairing of the terminal nucleotide.

19. A nucleic acid template as claimed in claim 4 wherein the sequestered end involves including the terminal nucleotide in an ITR structure with a double stranded D region.

20. The method of manufacturing single stranded nucleic acid molecules with sequestered ends as claimed in claim 11, wherein the template is as described in claim 4.


SEQUENCE LISTING (11 sequences)

SEQ ID NO: 1 - DNA, 3754 bp, Artificial Sequence
  Template A
gtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcaagcttctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtcagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactacttaagtgtacatatcagcacacaatagtccattatacgcgcgtataatgggcaattgtgtgctgatatgtacaggtcagactctgcgatcgcagagtctgacccgctgaggagatctacatgttctagaggatccgagcaaaaggccagcaaaaggccaggaaccgtaaaaaggccgcgttgctggcgtttttccataggctccgcccccctgacaagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttagcactctagaggatccgtgctattattgaagcatttatcagggttattgtctcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggaaattgtaagcgttaataattctagaggatcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatccagcgtatgcagccgccgcatagcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaatctagaggatcctcatcctgtctcttgatcagagcttgatcccctgcgccatcagatccttggcggcaagaaagccatccagtttactttgcagggcttcccaaccttaccagagggcgccccagctggcaattccggttcgcttgctgtccataaaaccgcccagtaatttcattgcggtcagactctgcgatcgcagagtctgaccactagttatcagcacacaatagtccattatacgcgcgtataatgggcaattgtgtgctgataactagtagatctgctagctaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggaattcttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagg

SEQ ID NO: 2 - DNA, 20 bp, adeno-associated virus 2
aggaacccctagtgatggag

SEQ ID NO: 3 - DNA, 20 bp, adeno-associated virus 1
gattacccctagtgatggag

SEQ ID NO: 4 - DNA, 20 bp, adeno-associated virus 3
ccatacctctagtgatggag

SEQ ID NO: 5 - DNA, 19 bp, adeno-associated virus 4
gcaaacctagatgatggag

SEQ ID NO: 6 - DNA, 19 bp, adeno-associated virus 5
cttgcttgagagtgtggag

SEQ ID NO: 7 - protein, 20 aa, Artificial Sequence
  Common binding motif
RGRGRGRGGGSGGSGGRGRG

SEQ ID NO: 8 - protein, 26 aa, Artificial Sequence
  RG rich domain
RRGDGRRRGGGGRGQGGRGRGGGFKG

SEQ ID NO: 9 - DNA, 114 bp, Artificial Sequence
  Figure 2 formatting element
cctcagcgggtcagactctgcgatcgcagagtctgacctgtacatatcagcacacaatagtccattatacgcgcgtataatggactattgtgtgctgatatgtacacttaagta

SEQ ID NO: 10 - DNA, 147 bp, adeno-associated virus 2
aaccggtgagggagagacgcgcggagcgagcgagtgactccggcccgctggtttccagcgggctgcgggcccgaaacgggcccgccggagtcactcgctcgctcggcgcgtctctccctcaccggttgaggtagtgatccccaagga

SEQ ID NO: 11 - DNA, 24 bp, Artificial Sequence
  Quadruplex motif - various organisms
ttagggttagggttagggttaggg